SEQUENCE LISTING (10 sequences)

SEQ ID NO: 1 - protein, 135 aa, Homo sapiens
GPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPA

SEQ ID NO: 2 - protein, 141 aa, Mus musculus
QGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTS

SEQ ID NO: 3 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 4 - protein, 381 aa, Homo sapiens
GPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 5 - protein, 410 aa, Mus musculus
MRCAPTAGAALVLCAATAGLLSAQGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKRLPKMTQLIGLTPNATHLHRPPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAAS

SEQ ID NO: 6 - protein, 387 aa, Mus musculus
QGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKRLPKMTQLIGLTPNATHLHRPPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAAS

SEQ ID NO: 7 - protein, 160 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPA

SEQ ID NO: 8 - protein, 164 aa, Mus musculus
MRCAPTAGAALVLCAATAGLLSAQGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTS

SEQ ID NO: 9 - protein, 25 aa, Homo sapiens
QSKSPRFASWDEMNVLAHGLLQLGQ

SEQ ID NO: 10 - protein, 25 aa, Mus musculus
QPEPPRFASWDEMNLLAHGLLQLGH

(19) United States
(12) Patent Application Publication
(10) Pub. No.: US 2022/0296681 A1
(43) Pub. Date: Sep. 22, 2022

(54) INHIBITION OF METASTASIS DEVELOPMENT BY NANGPTL-4

(71) Applicants: Deutsches Krebsforschungszentrum, Heidelberg (DE); Universität Heidelberg, Heidelberg (DE)

(72) Inventors: Hellmut G. Augustin, Heidelberg (DE); Corinne Hübers, Heidelberg (DE); Ashik Ahmed Abdul Pari, Heidelberg (DE); Martin Petkov, Heidelberg (DE); Moritz Felcht, Heidelberg (DE)

(21) Appl. No.: 17/633,351

(22) PCT Filed: Aug. 6, 2020
(86) PCT No.: PCT/EP2020/072179

§ 371 Date: Feb. 7, 2022

(30) Foreign Application Priority Data
Aug. 7, 2019 (EP) 19190563.7

(51) Int. Cl. A61K 38/18; G01N 33/574

(52) U.S. Cl. CPC A61K 38/1891; G01N 33/574

(57) ABSTRACT

The present invention relates to an N-terminal fragment of an angiopoietin like 4 (ANGPTL4) polypeptide, or a therapeutically active variant thereof, for use in the treatment of cancer in a subject. Moreover, the present invention provides an agent which increases the amount of an N-terminal fragment of an angiopoietin like 4 (ANGPTL4) polypeptide for use in treating cancer. Further, encompassed by the present invention is a method for identifying a candidate compound for the treatment of cancer.

The present invention relates to an N-terminal fragment of an angiopoietin like 4 (ANGPTL4) polypeptide, or a therapeutically active variant thereof, for use in the treatment of cancer in a subject. Moreover, the present invention provides an agent which increases the amount of an N-terminal fragment of an angiopoietin like 4 (ANGPTL4) polypeptide for use in treating cancer. Further, encompassed by the present invention is a method for identifying a candidate compound for the treatment of cancer.

### BACKGROUND SECTION

Cancer is the second leading cause of death worldwide and the disease is responsible for an estimated 9.6 million deaths in 2018. However, primary tumors can be controlled in the developed countries in most cases, whereas most patients succumb to the formation of distant metastases. Despite the clinical relevance of metastasis, the pathomechanisms hereof have so far not been unraveled in great molecular and mechanistic detail. Classically, metastasis formation is seen as a stepwise process of invasion of tumor cells into peritumoral tissue, intravasation into blood and lymphatic vessels, the ability to survive in the circulation (protection from anoikis), adhesion, extravasation at distant sites and macroscopic colony formation (Chiang et al.). While the early steps of the metastatic cascade are relatively common events during tumorigenesis, the formation of macroscopic colonies at distant sites is less common but most important for disease outcome.

During tumorigenesis, angiogenesis, the development of new blood vessels, is required for primary tumor growth beyond 100-150 μm size as well as for metastatic colonization, a late step in the metastatic cascade (Augustin et al., 2017). The development of a vascular network is controlled by a plethora of angiogenic growth factors. Vascular endothelial growth factor (VEGF) stands high in hierarchy of events that leads to the growth of blood vessels. Drugs blocking the VEGF/VEGFR pathway have therefore received wide application not just in Oncology, but also in Ophthalmology. In fact, some of the VEGF pathway blocking drugs (e.g. Bevacizumab) have emerged among the heaviest selling blockbuster drugs on this planet. Nevertheless, the clinical efficacy of VEGF pathway inhibiting drugs is limited and these drugs do not mediate long-term curative benefits (Folkman et al., Vasudev et al.). Overall, a better understanding of the relevance of angiogenesis and angiogenic factors during tumorigenesis and metastasis formation is urgently required.

The Angiopoietins (Ang) ligands and the related Angiopoietin-like proteins (ANGPTL) are another key regulatory system of angiogenesis. Angiopoietins and ANGPTL have a C-terminal fibrinogen-like domain and an N-terminal coiled-coil domain, except for ANGPTL-8, which is considered an atypical family member (Augustin et al., 2009). Angpt-1 and Angpt-2 bind the Tie2 receptor and some integrins. In contrast, ANGPTLs do not interact with Tie receptors, but are known to bind integrins and syndecans (Kirsch et al., 2017).

Most studies analyzing ANGPTL performed so far have concentrated on ANGPTL-4. However, the majority of these studies does not focus on the role of ANGPTL-4 during tumorigenesis/metastasis formation, but have been aimed at unraveling the roles of ANPGTL-4 in regulating fat metabolism. These studies could demonstrate that ANGPTL4-induced fat metabolic effects are strictly regulated by cleavage into a C-terminal fragment (cANGPTL-4) and an N-terminal fragment (nAngptl-4). nANGPTL-4 acts as an inhibitor of lipoprotein lipase (LPL) and thereby controls fat metabolism. Proteolytic cleavage of ANGPTL-4 is induced by proteinconvertases (furin, PCSK3, PC 5/6), which are localized in the subendothelial space as well as in the liver (Zhu et al., 2012).

It has also been established in recent years that tumors release ANGPTL-4 and thereby modulate angiogenesis as well as different steps of the metastatic cascade. However, results are controversial and even contradictory (reviewed by Tan et al., or Paglia et al.). Both, stimulating (Padua et al., Tian et al., Zhu et al. (2011), Huang et al., Hu et al., Liao et al., Baba et al., Zhang et al., Chen et al.) as well as inhibiting effects (Galaup et al. (2006), Cazes et al., 2006, Ito et al., Gomez Perdiguero, Okochi-Takada et al., Ng. et al.) of ANGPTL-4 on angiogenesis and tumor growth have been reported. Notably, cANGPTL-4 has been reported to induce vascular permeability via its interaction with integrin α5β1, followed by declustering and internalization of endothelial junction members like vascular endothelial cadherin (VE-Cadherin) and Claudin-5 (Zhu et al. (2011), Huang et al., Teo et al., Li et al.). Furthermore, cANGPTL-4 is reported to interact with integrins β1 and β5, which allows the activation of the pro-survival pathways phosphatidylinositol-3-kinase (PI3K)/protein kinase B (AKT) and extra-cellular signal regulated kinase (ERK) (Zhu et al. (2011)). ANGTPL-4-induced activation of these pathways protects from anoikis, one important steps in the metastatic cascade. These pro-metastatic effects are in line with the finding of an increase in cANGPTL-4 expression in the progression from a benign to a metastatic tumor state (Zhu et al. (2011),).

Thus, the reported state-of-the-art primarily portraits ANGPTL-4 as a pro-tumorigenic molecule. It is therefore of note that one earlier study reported that the serum contains mainly nANGPTL-4 and not the uncleaved form (flANGPTL-4) or cANGPTL-4. Moreover, nANGPTL-4 levels can pharmacologically be enhanced by fenofibrate treatment (Mandard et al.).

Zhang et al. investigated whether flANGPTL4, nANGPTL4 or cANGPTL4 have an effect on the tumorigenic properties of MG-63 cells (Zhang et al.: Angiopoietin-like 4 promotes osteosarcoma cell proliferation and migration and stimulates osteoclastogenesis. BMC Cancer (2018) 18:536). It was observed that no individual isoform had a strong effect on the tumorigenic properties. However, flANGPTL4 induced MG-63 cell proliferation and nANGPTL4 induced MG-63 cell migration.

WO2011/046515A1 discloses that antagonists to angiopoietin like 4 protein (ANGPTL4) can be used as antiproliferative agents.

WO2014027959A1 discloses an antibody that binds C terminal region of angiopoietin like 4 protein for the treatment of cancer.

Given the state of the art and the conflicting literature data, the inventor's work was aimed at assessing the role of ANGPTL-4 and particularly the cleavage products in tumor progression. It was shown for the first time that the ANGPTL-4 has contextual different (opposing) effects on angiogenesis and tumor growth. Confirming published data, both the full length protein and the C-Terminal domain stimulate angiogenesis in vitro and in vivo and thereby enhance tumor growth. Yet, the N-terminal fragment was identified to inhibit tumor angiogenesis and subsequently metastatic growth. The study has thereby not just identified the molecular mechanism of a long hypothesized pathomechanism (i.e., stimulation of primary tumor growth and inhibition of metastatic growth by the same molecule). The study has also identified an endogenous biomolecule, nANGPTL4, with potential immediate therapeutic potential.

In contrast to the C-terminal fragment of ANGPTL4, or to the full-length ANGPTL4, nANGPTL4 has the advantage that no angiogenic effect and no growth-enhancing effect on the primary tumor were found in the studies underlying the present invention. To our knowledge, there is currently no treatment which specifically inhibits the development of metastases.

The observed mechanism might be a protection mechanism for inhibiting the growth of metastases. In the past, it has been observed that the removal of a primary tumor can result in a massive development of metastases. The findings made in the studies underlying the present invention might explain this observation.

### BRIEF SUMMARY OF THE PRESENT INVENTION—EMBODIMENTS

The present invention relates to an active agent for use in the treatment of cancer in a subject. In an embodiment, the active agent is an N-terminal fragment of an angiopoietin-like 4 (ANGPTL4) polypeptide, or a therapeutically active variant thereof. In an alternative embodiment, the active agent is an agent, which increases the amount of an N-terminal fragment of an angiopoietin like 4 (ANGPTL4) polypeptide.

The invention further provides a method for the treatment of cancer, comprising administering a therapeutically effective amount of the active agent as set forth above, to a subject suffering from cancer.

The invention also relates to the use of an active agent as set forth above for the manufacture of a medicament for the treatment of cancer.

In an embodiment of the active agent, the method, or the use of the present invention, the treatment of cancer is the inhibition of metastasis development.

In an embodiment of the active agent, the method, or the use of the present invention, the inhibition of metastasis development is the inhibition of metastasis development after surgical removal of a tumor.

In an embodiment of the active agent, the method, or the use of the present invention, the active agent is administered perioperatively.

In an embodiment of the active agent, the method, or the use of the present invention, the active agent is administered before and/or after surgical removal of the tumor.

In an embodiment of the active agent, the method, or the use of the present invention, the active agent, the tumor is a primary tumor and the subject has not developed metastases at the time of the surgical removal of the tumor.

In an embodiment of the active agent, the method, or the use of the present invention, the active agent is administered intravenously or intraperitoneally.

In an embodiment of the active agent, the method, or the use of the present invention, the active agent is administered wherein the subject is a human subject and the angiopoietin like 4 (ANGPTL4) polypeptide is human ANGPTL4.

In an embodiment of the active agent, the method, or the use of the present invention, the N-terminal fragment, or therapeutically active variant thereof, is selected from
a) a polypeptide comprising or consisting of a sequence as shown in SEQ ID NO: 1, SEQ ID NO 2, SEQ ID NO 7 or SEQ ID NO 8,
b) a subfragment of the polypeptide of a), wherein the subfragment has a length of at least 50 amino acids, and
c) a polypeptide having at least 60%, at least 70%, at least 80%, at least 85%, at least 90%, at least 95%, at least 97%, at least 98% or at least 99% sequence identity to the polypeptide of a) or the subfragment of b).

In some embodiments, the active agent, the method, or the use of the present invention, the N-terminal fragment, or therapeutically active variant thereof, comprises a coiled-coil domain (CCD), but lacks a fibrinogen-like domain (FLD).

In an embodiment of the active agent, the method, or the use of the present invention, the N-terminal fragment, or therapeutically active variant thereof, is capable of
a) decreasing the activity of lipoprotein lipase,
b) binding to Syndecan-4, and/or
c) forming oligomers.

In an embodiment of the active agent, the method, or the use of the present invention, the cancer is selected from melanoma, breast cancer, colorectal cancer, ovarian cancer, renal cancer, primary cutaneous lymphomas, gastrointestinal cancer, lung cancer and hepatocellular carcinoma.

The present invention further relates to a method for identifying a candidate compound for the treatment of cancer, comprising
a) determining the amount of an N-terminal fragment of an angiopoietin like 4 (ANGPTL4) polypeptide in a first sample from a subject, wherein said first sample has been obtained from the subject prior to contacting the subject with the candidate compound,
b) determining the amount of the N-terminal fragment of an angiopoietin like 4 (ANGPTL4) polypeptide in a first sample from a subject, wherein said second sample has been obtained after contacting the subject with the candidate compound, and
c) comparing the amount of the N-terminal fragment of an angiopoietin like 4 (ANGPTL4) polypeptide in the second sample to the amount in the first sample, wherein an increased amount in the second sample as compared to the amount in the first sample is indicative for a candidate compound for the treatment of cancer.

In an embodiment of the aforementioned method for identifying a candidate compound, the sample is blood, serum or plasma sample.

The present invention also relates to the in vitro use of a detection agent for an N-terminal fragment of an angiopoietin like 4 (ANGPTL4) polypeptide for identifying a candidate compound for the treatment of cancer.

### DETAILED DESCRIPTION—DEFINITIONS

As set forth above, the present invention relates to an agent for use in the treatment of cancer in a subject. In a preferred embodiment, the agent is an N-terminal fragment of an angiopoietin like 4 (ANGPTL4) polypeptide, or a therapeutically active variant thereof. In another preferred embodiment, the agent is an agent which increases the amount of an N-terminal fragment of an angiopoietin like 4 (ANGPTL4) polypeptide.

The term “cancer”, as used herein, relates to a disease of an animal, including man, characterized by uncontrolled growth by a group of body cells (“cancer cells”). Preferably, the cancer is a cancer that can metastasize. More preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, dermatofibrosarcoma protuberans, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, primary cutaneous B-cell lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer. In particular, the cancer is selected from melanoma, breast cancer, colorectal cancer, ovarian cancer, renal cancer, primary cutaneous lymphomas, gastrointestinal cancer, lung cancer and hepatocellular carcinoma.

In some embodiments, the cancer is melanoma.

In some embodiments, the cancer is breast cancer.

In some embodiments, the cancer is colorectal cancer.

In some embodiments, the cancer is renal cancer.

In some embodiments, the cancer is ovarian cancer.

In some embodiments, the cancer is primary cutaneous lymphomas.

In some embodiments, the cancer is gastrointestinal cancer.

In some embodiments, the cancer is lung cancer.

In some embodiments, the cancer is hepatocellular carcinoma.

The terms “subject” and “patient” are used interchangeably herein. The subject to be treated in accordance with the present invention shall suffer from cancer. Accordingly, the subject is a cancer patient. According to some embodiments, the subject is a mammal, in particular a human, a rodent, a mouse, rat, a pig, monkey or guinea pig. In a preferred embodiment, the subject is a human subject suffering from cancer. In another preferred embodiment, the subject is a mouse.

In a preferred embodiment of present invention, the subject to be treated is a subject at risk of developing metastases. For example, the surgical removal of a tumor, in particular of a primary tumor is associated with a risk of developing metastases. Accordingly, the subject may be a subject suffering from cancer who will be subjected to a surgical removal of a tumor (in particular of a primary tumor) in the near future or a subject suffering from cancer whose tumor (in particular of a primary tumor) has been recently removed by surgery.

In an embodiment, the subject has not yet developed metastases. In particular, the subject has not yet developed overt metastases. For example, the subject has not developed metastases, such as overt metastases, at the time of the surgical removal of the tumor, in particular at the time of the surgical removal of the primary tumor.

In another embodiment, the subject has already developed metastases.

The term “treatment of cancer” as used herein refers to abrogating, inhibiting, slowing or reversing the progression of a cancer, ameliorating clinical symptoms of a cancer or preventing the appearance of clinical symptoms of cancer. The term also encompasses lengthening of the survival period of the subject undergoing treatment and/or lengthening the time of diseases progression.

It has been found in the studies underlying the invention that the agent as referred to herein, such as nANGPTL, inhibits metastasis formation at distant sites from the primary tumor. Thus, the agent is anti-metastatic drug. Accordingly, the term “treatment of cancer”, preferably, refers to the inhibition of metastasis development.

The terms “metastasis” and “metastasis development” are used interchangeably herein and relate to the growth of cancerous cells derived from a primary tumor located in one organ or tissue, in another, non-adjacent organ or tissue (i.e. at distant sites). In metastasis, cancer cells form one or more secondary tumors in other organs or tissues of the body. Preferably, the terms “metastasis” and “metastasis development” do not encompass cancer invasion, i.e. the direct extension and penetration by cancer cells into neighboring tissues.

The term “inhibiting metastasis development” as used herein, preferably, refers to slowing or even completely inhibiting the development of metastases, i.e. the development of new metastases. In one embodiment, the term refers to the prevention of metastasis development. Thus, the formation of new metastases is prevented. In another embodiment, the term refers to the reduction of metastasis development, i.e. to the reduction of newly formed metastases. In particular, the number of newly formed metastases is reduced as compared to the number of newly formed metastases in an untreated control subject, or as compared to the average number of newly formed metastases in a group of untreated control subjects. A control subject as referred to herein is, preferably, a subject who is not treated with the active agent as referred herein (such as the N-terminal fragment of the angiopoietin like 4 polypeptide, or a therapeutically active variant thereof).

Accordingly, a subject who is treated with an active agent as referred herein preferably develops a lower number of metastases as compared to the number of newly formed metastases in an untreated control subject, or as compared to the average number of newly formed metastases in a group of untreated control subjects. In some embodiments, the number of newly formed metastases is reduced by at least 50%. In some embodiments, the number of newly formed metastases is reduced by at least 60%. In some embodiments, the number of newly formed metastases is reduced by at least 70%. In some embodiments, the number of newly formed metastases is reduced by at least 80%. In some embodiments, the number of newly formed metastases is reduced by at least 90%. In some embodiments, the formation of new metastases is completely inhibited.

A reduction or prevention of metastasis development can be assessed by standard methods such as imaging methods. It is to be understood that the inhibition of metastasis development shall be a consequence of the treatment with the active agent as referred to herein.

In a preferred embodiment of the present invention, the inhibition of metastasis development is the inhibition of metastasis development in the treated subject after surgical removal of a tumor. Thus, the development of metastases after removal of the tumor by surgery is inhibited. In this surgery, the tumor is physically removed from the subject's body. Preferably, the entire tumor is removed. However, it is also envisaged that a portion of the tumor is removed, such as at least 70, 80, 90 or 95 wt % of the tumor.

The tumor that is removed by surgery is, preferably, a primary tumor, and, in particular, a primary malignant tumor. Further, it is envisaged that the primary tumor is a solid tumor. A primary tumor is a tumor growing at the anatomical site where tumor progression began and proceeded to yield a cancerous mass. Most cancers develop at their primary site but then can go on to metastasize or spread to other parts of the body. As set forth above, these further tumors are secondary tumors.

The active agent as referred to herein, such as the N-terminal fragment of the angiopoietin like 4 (ANGPTL4) polypeptide, or the therapeutically active variant thereof, may be administered by any method deemed appropriate. Preferably, the route of administration shall allow for delivering the active agent into the bloodstream of the treated subject. Accordingly, the active agent as referred to herein is preferably administered systemically, i.e. it is administered in to the systemic blood circulation. The active agent is preferably not administered locally to the tumor.

In a preferred embodiment, the active agent is administered parenterally. Parental administration includes, but is not limited to, intravenous, intraarterial, subcutaneous, intraperitoneal or intramuscular injection or infusion. In a particularly preferred embodiment, the active agent is administered intravenously. In another preferred embodiment, the active agent is administered intraperitoneally.

In accordance with the present invention, an effective amount of the active agent as referred to herein, such as the N-terminal fragment of the angiopoietin like 4 (ANGPTL4) polypeptide, or the therapeutically active variant thereof shall be administered. The term “effective amount”, as used herein, represents an amount of compound that allows for the treatment of cancer, and in particular that allows for inhibition of metastasis development (when administered to a subject suffering from cancer as described herein). In some embodiments, the N-terminal fragment, or therapeutically active variant thereof is administered at a dose in the range from 0.05 to 10 mg/kg body weight, such as from 0.1 to 3 mg/kg body weight. The administration can be e.g. three times a week, two times a week, once weekly, every second week, every third week or once a month.

If metastasis development after surgical removal of a tumor shall be inhibited, the active agent as referred to herein, such as the N-terminal fragment, or therapeutically active variant thereof is typically administered such that the patient's blood level of the active agent allows for inhibiting the treatment of cancer e.g. for inhibition of metastasis development. The active agent is preferably administered perioperatively, i.e. is administered within the perioperative period of the surgery. The perioperative period refers to the time period before, during and after surgical removal of the tumor. In some embodiments, the perioperative period refers to a period beginning 2-10 days before the surgery and ending about one month after said surgery. For example, the perioperative period refers to a period beginning 1-5 days prior to surgery and ending about one month following surgery. For example, the perioperative period refers to a period beginning 2 days prior to surgery and ending about 14 days following surgery. However, it is also envisaged to administer the agent for longer periods after surgery.

In some embodiments, the active agent is administered to the subject before surgery, i.e. prior to the start of surgery). If the active agent is administered before surgery, the active agent is, preferably, administered within a period of ten days, more preferably within a period of five days, and most preferably, within a period of two days prior to the surgery. Also preferably, the active agent is administered within a period of 24 hours before the start of therapy.

In some embodiments, the active agent is administered to the subject during the surgery, i.e. within the period from the start of the surgery to the end of the surgery.

In some embodiments, the active agent is administered to the subject after the surgery, i.e. after the end of the surgery. If the active agent is administered after surgery, the active agent is, preferably, administered within a period of at least three days, more preferably, within a period of at least one week, even more preferably within a period of at least two weeks, and most preferably, within a period of at least one month after the surgery.

In some embodiments, the active agent is administered to the subject before and after the surgery. Preferred periods before and after surgery are disclosed above.

The active agent may be administered once or more than once within the above periods. Depending on the length of the period, the agent is administered repeatedly.

In a preferred embodiment of the present invention, the agent is an N-terminal fragment of an angiopoietin like 4 (ANGPTL4) polypeptide, or a therapeutically active variant thereof.

The term “angiopoietin like 4 polypeptide” (abbreviated ANGPTL4) as used herein refers to an angiopoietin like 4 protein from a vertebrate. For example, it refers to a mammalian angiopoietin like 4 protein, such as a human, rodent, mouse, rat, porcine, bovine, monkey or guinea pig angiopoietin like 4 protein. Synonyms for ANGPTL4 are ARP4, FIAF, HFARP, NL2, PGAR and pp1158. Preferably, an ANGPTL4 polypeptide as referred to herein comprises an N-terminal coiled-coil domain (CCD) connected to a C-terminal fibrinogen-like domain (FLD) via a cleavable linker. The linker is herein also referred to as “cleavage site”. Typically, ANGPTL4 is proteolytically cleaved by proteinconvertases such as furin, or pro-protein-convertase 5/6 and 7 (as disclosed in Lei et al. J Biol Chem. 2011 May 6; 286(18): 15747-15756). As described elsewhere herein, the N-terminal fragment, or variant thereof, preferably, comprises a coiled-coil domain. Preferably, it does not comprise the C-terminal fragment, in particular, the C-terminal fragment after the cleavage site. Accordingly, the N-terminal fragment, or variant thereof, preferably, comprises a coiled-coil domain (CCD), but lacks a fibrinogen-like domain (FLD). In some embodiments, it lacks the entire fibrinogen-like domain of an ANGPTL4 polypeptide.

In a preferred embodiment, the angiopoietin like 4 polypeptide is a human angiopoietin like 4 polypeptide. The sequence of the human angiopoietin like 4 polypeptide is well known in the art and is e.g. disclosed in Hato et al. (Trends Cardiovasc Med. 2008 January; 18(1):6-14) which herewith is incorporated by reference with respect to the sequence.

The unprocessed human ANGPTL4 has a length of 406 amino acids. The amino acid sequence of the unprocessed human ANGPTL4 is as follows:

SEQ ID NO: 3 (human ANGPTL4 with signal peptide (two underlines), N-terminal domain (bold), cleavage site (RRKR, underlined) and C-terminal domain (italics):

[symbol]

GPVQSKSPRFASWDEMNVL
AHGLLQLGQGLREHAERTRSQLSALERRLSACGSACQGTEGSTDLPLAP
ESRVDPEVLHSLQTQLKAQNSRIQQLFHKVAQQQRHLEKQHLRIQHLQS
QFGLLDHKHLDHEVAKPARRKRLPEMAQPVDPAHNVSRLHRLPRDCQEL
FQVGERQSGLFEIQPQGSPPFLVNCKMTSDGGWTVIQRRHDGSVDFNRP
WEAYKAGFGDPHGEFWLGLEKVHSITGDRNSRLAVQLRDWDGNAELLQF
SVHLGGEDTAYSLQLTAPVAGQLGATTVPPSGLSVPFSTWDQDHDLRRD
KNCAKSLSGGWWFGTCSHSNLNGQYFRSIPQQRQKLKKGIFWKTWRGRY
YPLQATTMLIQPMAAEAAS

The first 25 amino acids of SEQ ID NO: 3 form a signal peptide which is cleaved off to yield the mature human ANGPTL4 polypeptide which has the following sequence:

SEQ ID NO: 4 (human ANGPTL4 without signal peptide, but with N-terminal domain (bold), cleavage site (RRKR, underlined) and C-terminal domain (italics).

GPVQSKSPRFASWDEMNVL
AHGLLQLGQGLREHAERTRSQLSALERRLSACGSACQGTEGSTDLPLAP
ESRVDPEVLHSLQTQLKAQNSRIQQLFHKVAQQQRHLEKQHLRIQHLQS
QFGLLDHKHLDHEVAKPARRKRLPEMAQPVDPAHNVSRLHRLPRDCQEL
FQVGERQSGLFEIQPQGSPPFLVNCKMTSDGGWTVIQRRHDGSVDFNRP
WEAYKAGFGDPHGEFWLGLEKVHSITGDRNSRLAVQLRDWDGNAELLQF
SVHLGGEDTAYSLQLTAPVAGQLGATTVPPSGLSVPFSTWDQDHDLRRD
KNCAKSLSGGWWFGTCSHSNLNGQYFRSIPQQRQKLKKGIFWKTWRGRY
YPLQATTMLIQPMAAEAAS

After secretion, the mature ANGPTL4 is proteolytically cleaved at the cleavage site (RRKR) giving rise to an N-terminal coiled-coil fragment (nANGPTL4) and a C-terminal fibrinogen-like domain (cANGPTL4).

The N-terminal fragment of the human angiopoietin like 4 (ANGPTL4) polypeptide preferably has a sequence as shown in SEQ ID NO: 1:

SEQ ID NO: 1 (N-terminal fragment of the human angiopoietin like 4 (ANGPTL4) polypeptide):

GPVQSKSPRFASWDEMNVL
AHGLLQLGQGLREHAERTRSQLSALERRLSACGSACQGTEGSTDLPLAP
ESRVDPEVLHSLQTQLKAQNSRIQQLFHKVAQQQRHLEKQHLRIQHLQS
QFGLLDHKHLDHEVAKPA

In another preferred embodiment, the angiopoietin like 4 polypeptide is a mouse angiopoietin like 4 polypeptide. The sequence of the mouse angiopoietin like 4 polypeptide is well known in the art and is e.g. disclosed in Lee et al., 2009 JBC 284: 13735-45.

The unprocessed mouse ANGPTL4 has a length of 410 amino acids. The amino acid sequence of the unprocessed mouse ANGPTL4 is as follows:

SEQ ID NO: 5 (ANGPTL4 with signal peptide (two underlines), N-terminal domain (bold), cleavage site (RGKR, underlined) and C-terminal domain (italics):

MRCAPTAGAALVLCAATAGLLSAQGRPAQPEPPRFASWDEMNLL
AHGLLQLGHGLREHVERTRGQLGALERRMAACGNACQGPKGKDAPFKDS
EDRVPEGQTPETLQSLQTQLKAQNSKIQQLFQKVAQQQRYLSKQNLRIQ
NLQSQIDLLAPTHLDNGVDKTSRGKRLPKAITQLIGLTPNATHLHRPPR
DCQELFQEGERHSGLFQIQPLGSPPFLVNCEMTSDGGWTVIQRRLNGSV
DFNQSWEAYKDGFGDPQGEFWLGLEKAKISITGNRGSQLAVQLQDWDGN
AKLLQFPIHLGGEDTAYSLQLTEPTANELGATNVSPNGLSLPFSTWDQD
HDLRGDLNCAKSLSGGWWFGTCSHSNLNGQYFHSIPRQRQERKKGIFWK
TWKGRYYPLQATTLLIQPMEATAAS*

The first 23 amino acids of SEQ ID NO: 5 form a signal peptide which is cleaved off to yield the mature mouse ANGPTL4 polypeptide which has the following sequence:

SEQ ID NO: 6 (mouse ANGPTL4 without signal peptide, but with N-terminal domain (bold), cleavage site (RGKR, underlined) and C-terminal domain (italics):

QGRPAQPEPPRFASWDEMNLL
AHGLLQLGHGLREHVERTRGQLGALERRMAACGNACQGPKGKDAPFKDS
EDRVPEGQTPETLQSLQTQLKAQNSKIQQLFQKVAQQQRYLSKQNLRIQ
NLQSQIDLLAPTHLDNGVDKTSRGKRLPKMTQLIGLTPNATHLHRPPRD
CQELFQEGERHSGLFQIQPLGSPPFLVNCEMTSDGGWTVIQRRLNGSVD
FNQSWEAYKDGFGDPQGEFWLGLEKAKISITGNRGSQLAVQLQDWDGNA
KLLQFPIHLGGEDTAYSLQLTEPTANELGATNVSPNGLSLPFSTWDQDH
DLRGDLNCAKSLSGGWWFGTCSHSNLNGQYFHSIPRQRQERKKGIFWKT
WKGRYYPLQATTLLIQPMEATAAS*

After secretion, the mature ANGPTL4 is proteolytically cleaved at the cleavage site (RGKR) giving rise to an N-terminal coiled-coil fragment (nANGPTL4) and a C-terminal fibrinogen-like domain (cANGPTL4).

The N-terminal fragment of the mouse angiopoietin like 4 (ANGPTL4) polypeptide preferably has a sequence as shown in SEQ ID NO: 2:

SEQ ID NO: 2 (N-terminal fragment of the mouse angiopoietin like 4 (ANGPTL4) polypeptide):

QGRPAQPEPPRFASWDEMNLL
AHGLLQLGHGLREHVERTRGQLGALERRMAACGNACQGPKGKDAPFKDS
EDRVPEGQTPETLQSLQTQLKAQNSKIQQLFQKVAQQQRYLSKQNLRIQ
NLQSQIDLLAPTHLDNGVDKTS

Preferably, the N-terminal fragment of an angiopoietin like 4 (ANGPTL4) polypeptide as referred to herein has the amino acid sequence of a naturally occurring N-terminal fragment of ANGPTL4, such as a naturally occurring N-terminal fragment of human or mouse ANGPTL4, which is produced in a mammalian organism, such as in a mouse or a human by proteolytic cleavage at the cleavage site which is present between the N-terminal fragment and C-terminal fragment. Said cleavage sites are well-known in the art. Further, the mouse (RGKR) and the human (RRKR) cleavages sites are disclosed above. Preferably, the N-terminal fragment of an angiopoietin like 4 (ANGPTL4) polypeptide is a polypeptide comprising a sequence as shown in SEQ ID NO: 1 or SEQ ID NO 2.

Further, the N-terminal fragment may comprise the signal peptide of a ANGPTL4 polypeptide. The signal sequence of human and mouse is shown above (first 25 and 23 amino acids in the unprocessed sequence (SEQ ID NO 3 and 5, respectively).

Accordingly, the N-terminal fragment of an angiopoietin like 4 (ANGPTL4) polypeptide may be, in some embodiments, a polypeptide comprising a sequence as shown in SEQ ID NO: 7 or SEQ ID NO: 8. In some embodiments, it may be a polypeptide consisting of a sequence as shown in SEQ ID NO: 7 or SEQ ID NO: 8.

SEQ ID NO: 7 (human ANGPTL4 with signal peptide (two underlines), N-terminal domain (bold), but without cleavage site and without C-terminal domain:

[symbol]

GPVQSKSPRFASWDEMNVL
AHGLLQLGQGLREHAERTRSQLSALERRLSACGSACQGTEGSTDLPLAP
ESRVDPEVLHSLQTQLKAQNSRIQQLFHKVAQQQRHLEKQHLRIQHLQS
QFGLLDHKHLDHEVAKPA

SEQ ID NO: 8 (mouse ANGPTL4 with signal peptide (two underlines), N-terminal domain (bold), but without cleavage site and without C-terminal domain:

MRCAPTAGAALVLCAATAGLLSAQGRPAQPEPPRFASWDEMNLL
AHGLLQLGHGLREHVERTRGQLGALERRMAACGNACQGPKGKDAPFKDS
EDRVPEGQTPETLQSLQTQLKAQNSKIQQLFQKVAQQQRYLSKQNLRIQ
NLQSQIDLLAPTHLDNGVDKTS

SEQ ID NO: 8 was tested in the studies underlying the present invention.

Further, the N-terminal fragment may additionally comprise the first amino acid (R in human ANGPTL4), or the first and second amino acid (RR in human ANGPTL4) of the cleavage site.

It is to be understood that the N-terminal fragment of an angiopoietin like 4 (ANGPTL4) polypeptide, or a therapeutically active variant thereof, is not a full-length ANGPTL4 polypeptide. Rather, it should be a truncated ANGPTL4 polypeptide (or a variant thereof). The term “truncated” refers to a truncated ANGPTL4 polypeptide that lacks amino acids of at least the C-terminal region of a full-length ANGPTL4 polypeptide, and thus to an ANGPTL4 polypeptide having C terminal amino acids removed.

Accordingly, the N-terminal fragment of an angiopoietin like 4 (ANGPTL4) polypeptide, or a therapeutically active variant thereof as referred to herein, lacks the C-terminal fragment of an ANGPTL4 polypeptide, i.e. the entire C-terminal fragment, or a portion thereof. Preferably, the C-terminal fragment of an ANGPTL4 polypeptide comprises a fibrinogen-like domain. Accordingly, the N-terminal fragment, or variant thereof, as referred to herein, preferably, lacks a fibrinogen-like domain, i.e. does not comprise a fibrinogen-like domain. More preferably, the C-terminal fragment of an ANGPTL4 polypeptide is the fragment following the cleavage site (indicated in italics in SEQ ID NO 3 and 5 above). Accordingly, the N-terminal fragment, or variant thereof, as referred to herein, preferably, lacks the fragment following the cleavage site.

In some embodiments, the N-terminal fragment, or variant thereof, lacks the entire C-terminal fibrinogen-like domain of an ANGPTL4 polypeptide, i.e. does not comprise the fragment following the cleavage site. In some embodiments, the N-terminal fragment, or variant thereof, lacks at least 100, at least 200, or at least 250 amino acids of the C-terminus. For example, the N-terminal fragment, or variant thereof, may comprise the N-terminal fragment of the human or mouse ANGPTL4 polypeptide, but lack at least 100, at least 200, or at least 250 amino acids of the C-terminus of the human or mouse ANGPTL4 polypeptide, respectively. In some embodiments, the N-terminal fragment, or variant thereof, comprises not more than 50, 30 or 10 consecutive amino acids of the C-terminus of an ANGPTL4 polypeptide.

How to produce a truncated polypeptide is well known in the art. For example, it could be produced by expressing a polynucleotide which encodes for the truncated polypeptide in a host cell. Said polynucleotide can be produced by introducing an artificial stop codon at the position at which the polypeptide should be truncated. For example, an artificial stop codon can be introduced in a codon which encodes an amino acid within the cleavage site. Accordingly, an N-terminal fragment of an angiopoietin like 4 (ANGPTL4) polypeptide, or a therapeutically active variant thereof, is e.g. obtained or obtainable by truncating a ANGPTL4 polypeptide within the cleavage site. Alternatively, an N-terminal fragment of an angiopoietin like 4 (ANGPTL4) polypeptide, or a therapeutically active variant thereof, is e.g. obtained or obtainable by truncating a ANGPTL4 polypeptide within the cleavage site, or close to the cleavage site, such as within the region starting at amino acid position 155 of SEQ ID NO 3 and ending at amino acid position 163 of SEQ ID NO: 3, or within the region starting at amino acid position 140 of SEQ ID NO 3 and ending at amino acid position 163 of SEQ ID NO: 3.

As set forth above, the N-terminal fragment of an angiopoietin like 4 (ANGPTL4) polypeptide as referred to herein is, preferably, a polypeptide comprising an amino acid sequence as shown in SEQ ID NO: 1, SEQ ID NO: 2, or SEQ ID NO: 7 or SEQ ID NO: 8. Thus, the N-terminal fragment is a polypeptide. The term “polypeptide”, preferably, refers to an amino acid polymer in which one or more amino acid residue is an artificial chemical mimetic of a corresponding naturally occurring amino acid, as well as to a naturally occurring amino acid polymer and non-naturally occurring amino acid polymer. Unless otherwise indicated, a particular polypeptide sequence also implicitly encompasses conservatively modified variants thereof.

The present invention is not limited to the use of naturally occurring N-terminal fragments of an angiopoietin like 4 (ANGPTL4) polypeptide in the treatment of cancer. Rather, the present invention also encompasses the use of therapeutically active variants of an N-terminal fragment of an angiopoietin like 4 (ANGPTL4) polypeptide, in particular of therapeutically active variants of an N-terminal fragment of an angiopoietin like 4 (ANGPTL4) polypeptide as defined above.

Preferably, a variant as referred to herein shall be therapeutically active. Accordingly, a variant shall be capable of treating cancer. In particular, a variant shall be capable of inhibiting metastasis development in a subject suffering from cancer (as explained elsewhere herein).

Additionally or alternatively, the N-terminal fragment of an angiopoietin like 4 (ANGPTL4) polypeptide, or a therapeutically active variant thereof, is preferably, capable of decreasing, i.e. inhibiting, the activity of lipoprotein lipase (e.g. of mouse or human lipoprotein lipase). A lipoprotein lipase (3.1.1.34) preferably catalyzes the hydrolysis of triacylglycerol in chylomicrons and very low density lipoproteins (VLDLs) into diacylglycerol and a free fatty acid anion. In certain embodiments, a lipoprotein lipase is also able to hydrolyze diacylglycerol. Whether a compound is capable of decreasing the activity of lipoprotein lipase can be assessed by enzymatic assays. In an embodiment, it is assessed as described in U.S. Pat. No. 8,591,891B2.

Additionally or alternatively, the N-terminal fragment of an angiopoietin like 4 (ANGPTL4) polypeptide, or a therapeutically active variant thereof, is preferably, capable of binding to Syndecan-4, i.e. a Syndecan-4 polypeptide (e.g. to the human or mouse Syndecan-4 polypeptide). Whether a compound is capable of binding to Syndecan-4 can be determined by routine binding assays. In an embodiment, it is assessed as described in Kirsch et al. (Kirsch N, et al.: Angiopoietin-like 4 is a Wnt signaling antagonist that promotes LRP6 turnover. Dev Cell, 43: 71-82, 2017).

Additionally or alternatively, the N-terminal fragment of an angiopoietin like 4 (ANGPTL4) polypeptide, or a therapeutically active variant thereof, is preferably capable of forming oligomers, i.e. of forming oligomers of the N-terminal fragment, or variant thereof. In an embodiment, the oligomers are dimers and/or tetramers.

Further, the N-terminal fragment of an angiopoietin like 4 (ANGPTL4) polypeptide, or a therapeutically active variant thereof, is preferably capable of inhibiting angiogenesis. In particular, the fragment or variant thereof shall be capable of inhibiting VEGF (Vascular endothelial growth factor) induced angiogenesis. Whether a compound is capable of inhibiting angiogenesis, or not, can be assessed by well-known methods. E.g., it can be assessed as described in Example 5 below. Preferably, angiogenesis is inhibited at distant sites from the primary tumor.

In summary, the N-terminal fragment of an angiopoietin like 4 (ANGPTL4) polypeptide, or a therapeutically active variant thereof (such as the subfragment as referred to herein), has one or more of the following activities:
i. it is capable of decreasing the activity of lipoprotein lipase,
ii. it is capable of binding to Syndecan-4,
iii. it is capable of forming oligomers,
iv. it is capable of treating cancer,
v. it is capable of inhibiting angiogenesis.

Preferably, the N-terminal fragment of an angiopoietin like 4 (ANGPTL4) polypeptide, or a therapeutically active variant thereof, has said one or more activities when administered to a subject, preferably to a subject suffering from cancer as defined herein.

Further, it is envisaged that the therapeutically active variant of an N-terminal fragment of an angiopoietin like 4 (ANGPTL4) polypeptide is a subfragment of an N-terminal fragment of an ANGPTL4 polypeptide as defined herein, such as a subfragment of a polypeptide comprising an amino acid sequence as shown in SEQ ID NO: 1, SEQ ID NO 2, SEQ ID NO 7 or 8.

Further, it is envisaged that the therapeutically active variant of an N-terminal fragment of an angiopoietin like 4 (ANGPTL4) polypeptide is a polypeptide having at least 60%, at least 70%, at least 80%, at least 85%, at least 90%, at least 95%, at least 97%, at least 98% or at least 99% sequence identity to an N-terminal fragment of an ANGPTL4 polypeptide as defined herein, such as to a polypeptide comprising an amino acid sequence as shown in SEQ ID NO: 1, SEQ ID NO 2, SEQ ID NO 7 or 8, or to a subfragment thereof.

Accordingly, the N-terminal fragment, or therapeutically active variant thereof, is preferably selected from
(a) a polypeptide comprising a sequence as shown in SEQ ID NO: 1, SEQ ID NO 2, SEQ ID NO 7 or 8,
(b) a subfragment of the polypeptide of a), or
(c) a polypeptide having at least 60%, at least 70%, at least 80%, at least 85%, at least 90%, at least 95%, at least 97%, at least 98% or at least 99% sequence identity to the polypeptide of a) or to the subfragment of b).

In some embodiments, the subfragment has a length of at least 50 amino acids.

In some embodiments, the subfragment has a length of at least 60 amino acids.

In some embodiments, the subfragment has a length of at least 70 amino acids.

In some embodiments, the subfragment has a length of at least 80 amino acids.

In some embodiments, the subfragment has a length of at least 90 amino acids.

In some embodiments, the subfragment has a length of at least 100 amino acids.

In some embodiments, the subfragment has a length of at least 110 amino acids.

The term “identical” as used herein, preferably, refers to sequence identity characterized by determining the number of identical amino acids between amino acid sequences wherein the sequences are aligned so that the highest order match is obtained. For sequence comparison, typically one sequence acts as a reference sequence, to which test sequences are compared. When using a sequence comparison algorithm, test and refer-ence sequences are entered into a computer, subsequence coordinates are designated, if necessary, and sequence algorithm program parameters are designated. Preferably, de-fault program parameters, i.e. standard parameters can be used. The sequence comparison algorithm then calculates the percent sequence identities for the test sequences relative to the reference sequence, based on the program parameters. A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are opti-mally aligned.

The percent identity values are, preferably, calculated over the entire amino sequence region. A series of programs based on a variety of algorithms is available to the skilled worker for comparing different sequences. In a preferred embodiment, the percent identi-ty between two amino acid sequences is determined using the Needleman and Wunsch algorithm (Needleman 1970, J. Mol. Biol. (48):444-453) which has been incorporated into the needle program in the EMBOSS software package (EMBOSS: The European Mo-lecular Biology Open Software Suite, Rice, P., Longden, I., and Bleasby, A., Trends in Genetics 16(6), 276-277, 2000), a BLOSUM62 scoring matrix, and a gap opening penalty of 10 and a gap entension pentalty of 0.5. A preferred, non-limiting example of parame-ters to be used for aligning two amino acid sequences using the needle program are the default parameters, including the EBLOSUM62 scoring matrix, a gap opening penalty of 10 and a gap extension penalty of 0.5. In some embodiments, the percent identity between two amino acid sequences can also be determined using the algorithm of E. Meyers and W. Miller (Comput. Appl. Biosci., 4:1 1-17, 1988) which has been incorporated into the ALIGN program (version 2.0), using a PAM120 weight residue table, a gap length penalty of 12 and a gap penalty of 4.

In accordance with the present invention, it is envisaged that the N-terminal fragment of an angiopoietin like 4 polypeptide, or the therapeutically active variant thereof, may comprise a conserved SE1 motif. The SE1 (specific epitope 1) motif is a highly conserved consensus motif that is present within the coiled-coil domain (CCD) of both ANGPTL. The SE1 motif is well known in the art and e.g. described in Ge et al. 2004, J Lipid Res 45: 2071-7920 and Lee et al., 2009 JBC 284: 13735-45.

In an embodiment, the SE1 motif is the human SE1 motif (e.g. if the variant is a variant of human nANGPTL4). The human SE1 motif has a sequence as shown in SEQ ID NO: 9 (SKSPRFASWDEMNVLAHGLLQLGQ).

In an embodiment, the SE1 motif is the mouse SE1 motif (e.g. if the variant is a variant of human nANGPTL4). The mouse SE1 motif has a sequence as shown in SEQ ID NO: 10 (QPEPPRFASWDEMNLLAHGLLQLGH).

Accordingly, the fragment or variant as set forth herein may comprise a motif having a sequence shown in SEQ ID NO: 9 or 10.

In accordance with the present invention, the N-terminal fragment of an angiopoietin like 4 polypeptide, or a therapeutically active variant thereof, preferably comprises a coiled-coil domain (CCD). However, as set forth herein elsewhere, it shall lack a fibrinogen-like domain.

Preferably, the N-terminal fragment of an angiopoietin like 4 polypeptide, or a therapeutically active variant thereof, has a length of less than 250 amino acids, more preferably of less than 200 amino acids, and most preferably of less than 170 amino acids.

Preferably, the N-terminal fragment of an angiopoietin like 4 polypeptide, or a therapeutically active variant thereof, comprises conserved amino acids which correspond to E40, C76, C80, H46, E50 and E53 of the murine ANGPTL4 (siehe Yau et al., JBC, 2009; 284: 11942-52).

Preferably, the N-terminal fragment of an angiopoietin like 4 polypeptide, or a therapeutically active variant thereof, as referred to herein has been produced recombinantly in a host cell, i.e. by expressing a polynucleotide encoding said N-terminal fragment of the angiopoietin like 4 (ANGPTL4) polypeptide, or a therapeutically active variant thereof in host cell. Preferably, said host cell is a eukaryotic cell, more preferably, an animal cell, even more preferably, a vertebrate cell and most preferably, a mammalian cell. In some embodiments, the host cell is an insect cell. Mammalian host systems for the expression of recombinant proteins also are well known to those of skill in the art. Host cell strains may be chosen for a particular ability to process the expressed protein or produce certain posttranslation modifications that will be useful in providing protein activity. Such modifications of the polypeptide include, but are not limited to, acetylation, carboxylation, glycosylation, phosphorylation, lipidation and acylation. Different host cells such as Chinese hamster ovary (CHO) cell lines, COS cells (such as COS-7), HeLa cells, W138, BHK, HepG2, 3T3, RIN, MDCK, A549, PC12, K562 and 293 cells have specific cellular machinery and characteristic mechanisms for such post-translational activities and may be chosen to ensure the correct modification and processing of the introduced, foreign protein. In some embodiments, the N-terminal fragment, or variant thereof, is capable of forming disulfide-bridges (e.g. as disclosed in Ge et al. 2004, J Lipid Res 45: 2071-7920 or Yin et al. J Biol Chem. 2009 May 8; 284(19):13213-22). Such disulfide bridges may be formed to generate disulfide-linked dimers and tetramers of the N-terminal fragment or variants thereof.

As set forth above, the active agent to be administered can be an agent which increases the amount of an N-terminal fragment of an angiopoietin like 4 (ANGPTL4) polypeptide. Accordingly, the present invention relates to an agent which increases the amount of an N-terminal fragment of an angiopoietin like 4 (ANGPTL4) polypeptide for use in treating cancer, such as for inhibiting metastasis development. The definitions given above apply accordingly.

Preferably, said agent, when administered to a subject, increases the amount of N-terminal fragment of an angiopoietin like 4 (ANGPTL4) polypeptide in the blood circulation of the subject. More preferably, said increase is an increase of at least 20%, at least 50%, or at least 100% as compared to an untreated control subject.

In some embodiments, the agent which increases the amount of an N-terminal fragment of an angiopoietin like 4 (ANGPTL4) polypeptide is a fibrate, such as fenofibrate. In some embodiments, the agent is TGFβ (Tumor Growth Factor beta, such as human TGFβ1, TGFβ2, and TGFβ3. In some embodiments, the agent is a glucocorticoid.

The invention further provides a method for the treatment of cancer, comprising administering a therapeutically effective amount of the N-terminal fragment of an angiopoietin like 4 (ANGPTL4) polypeptide as defined herein, or a therapeutically active variant thereof, to a subject suffering from cancer.

The invention further provides a method for the treatment of cancer, comprising removing a tumor from a subject suffering from cancer by surgery and administering a therapeutically effective amount of the N-terminal fragment of an angiopoietin like 4 (ANGPTL4) polypeptide as defined herein, or a therapeutically active variant thereof.

The invention also relates to an N-terminal fragment of an angiopoietin like 4 (ANGPTL4) polypeptide as defined herein, or a therapeutically active variant thereof, for use as a medicament.

The N-terminal fragment of an angiopoietin like 4 (ANGPTL4) polypeptide as defined herein, or a therapeutically active variant thereof, shall be administered in an effective amount.

The invention also provides for the use of the N-terminal fragment of an angiopoietin like 4 (ANGPTL4) polypeptide as defined herein, or a therapeutically active variant thereof, for the manufacture of a medicament for the treatment of cancer.

The invention further relates to the use of an N-terminal fragment of an angiopoietin like 4 (ANGPTL4) polypeptide as defined herein, or a therapeutically active variant thereof, for the manufacture of a medicament for the treatment of cancer.

The definitions and explanations given herein above apply mutatis mutandis to the following method of the present invention.

The studies underlying the present invention indicate that a patient suffering from cancer might benefit from increased amounts of nANGPTL4 in the circulation. Specifically, increased amounts of nANGPTL4 in the circulation might inhibit the development of metastases in said patient. As nANGPTL4 is an endogenous biomolecule, it can be potentially used as a biomarker for identifying drug candidates for the treatment of cancer, such as drug candidates for the inhibition of metastasis.

Accordingly, the present invention relates to a method for identifying a candidate compound for the treatment of cancer, comprising
a) determining the amount of an N-terminal fragment of an angiopoietin like 4 (ANGPTL4) polypeptide in a first sample from a subject, wherein said first sample has been obtained from the subject prior to contacting the subject with the candidate compound,
b) determining the amount of the N-terminal fragment of an angiopoietin like 4 (ANGPTL4) polypeptide in a first sample from a subject, wherein said second sample has been obtained after contacting the subject with the candidate compound, and
c) comparing the amount of the N-terminal fragment of an angiopoietin like 4 (ANGPTL4) polypeptide in the second sample to the amount in the first sample.

Preferably, the identification of a candidate compound is based on the results of the comparison step (c). Thus, the above method of the present invention can comprise the further step (d) of identifying a candidate compound for the treatment of cancer based on the results of step (c).

The above method of the present invention includes a method which essentially consists of the aforementioned steps or a method which includes further steps. Moreover, the method of the present invention, preferably, is an in vitro method. It may comprise steps in addition to those explicitly mentioned above. For example, further steps may relate to sample pre-treatments, evaluation of the results obtained by the method, and the confirmation that an identified candidate allows for the treatment of cancer. The method may be carried out manually or assisted by automation. Preferably, step (a), (b) and/or (c) may in total or in part be assisted by automation, e.g., by a suitable robotic and sensory equipment for the determination in step (a) and (b) or a computer-implemented comparison in step (c).

The test subject is preferably an animal, more preferably, a vertebrate, and most preferably a mammal, such as a non-human mammal. Examples of suitable animals for use in the above screening method of the invention include, but are not limited to, mice, rats, rabbits, monkeys, guinea pigs, dogs and cats. The subject to be tested shall be contacted with the test compound, i.e. the candidate compound. According, the test compound is administered (e.g., orally, rectally or parenterally such as intraperitoneally or intravenously) to a suitable animal and its effect on nANGPTL4 is tested.

Examples of candidate compounds that can be tested according to the above method of the invention include, but are not limited to, nucleic acids (e.g., DNA and RNA), carbohydrates, lipids, proteins, peptides, peptidomimetics, small molecules and other drugs. Compounds can be obtained using any of the numerous approaches in combinatorial library methods known in the art.

In some embodiments, the candidate compound is a compound of low molecular weight (i.e. a small molecule) or the library is composed of molecules with low molecular weight (“small molecule library”). A “small molecule” is defined as a complex collection of compounds, which are produced in a non-biological way, meaning that they are not produced by recombinant expression, like for instance most protein or peptide libraries.

The compound to be tested for its suitability for the therapy of cancer can be formulated with a pharmaceutically acceptable carrier to produce a pharmaceutical composition, which can be administered to a human or other animal. A pharmaceutically-acceptable carrier can be, for example, water, sodium phosphate buffer, phosphate-buffered saline, normal saline or Ringer's solution or other physiologically-buffered saline, or other solvent or vehicle such as a glycol, glycerol, an oil such as olive oil or an injectable organic ester. A pharmaceutically acceptable carrier can also contain physiologically acceptable compounds that act, for example, to stabilize or increase the absorption of the modulatory compound. One skilled in the art would know that the choice of a pharmaceutically acceptable carrier, including a physiologically acceptable compound, depends, for example, on the route of administration of the composition.

The term “sample” refers to a sample of a body fluid, to a sample of separated cells or to a sample from a tissue or an organ. Samples of body fluids can be obtained by well-known techniques and include, samples of blood, plasma, serum, urine, lymphatic fluid, sputum, ascites, or any other bodily secretion or derivative thereof. Tissue or organ samples may be obtained from any tissue or organ by, e.g., biopsy. In a preferred embodiment, the sample is a blood, serum or a plasma sample.

In accordance with the aforementioned method of the present invention, the amount of nANGPTL4 (or a variant thereof) shall be determined in a first and a second sample from the subject. The “first sample” is understood as a sample which is obtained in order to reflect the amount of markers prior to the administration of the test compound. Accordingly, said first sample has been obtained from the subject prior to contacting the subject with the candidate compound. Preferably, the first sample is obtained within one month, more preferably, within one week, even more preferably, within three days, or most preferably, within 24 hours prior to contacting the subject with the candidate compound.

The “second sample” is, preferably, understood as a sample which is obtained in order to reflect a change of the amount of nANGPTL4 (or a variant thereof) as compared to the amount of the e marker in the first sample. Preferably, the second sample is obtained not too late after the subject has been contacted with the candidate compound, but also not too early after the first sample. Preferably, the second sample has been obtained within one week, even more preferably, within three days, or most preferably, within 24 hours after contacting the subject with the candidate compound.

The term “amount” as used herein encompasses the absolute amount of the N-terminal fragment of an angiopoietin like 4 (ANGPTL4) polypeptide (or variant thereof) as referred to herein, the relative amount or concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts measured from biological read out systems in response to the peptides or intensity signals obtained from specifically bound ligands. It is to be understood that values correlating to the aforementioned amounts or parameters can also be obtained by all standard mathematical operations.

Preferably, the amount of endogenous nANGPTL4 is determined in the first sample and second sample. For example, if the test subject is a mouse, the amount of endogenously produced mouse nANGPTL4 is determined.

The term “comparing” as used herein encompasses comparing the amount of the N-terminal fragment of an angiopoietin like 4 (ANGPTL4) polypeptide (or variant thereof) in the first sample with an amount of said marker in the second sample. It is to be understood that comparing as used herein refers to a comparison of corresponding parameters or values. Preferably, the identification of a candidate compound for the treatment of cancer is based on the comparison of the amount of the marker of the second sample to the amount of the marker in the first sample.

Preferably, an increased amount of the marker nANGPTL4 (or a variant thereof) in the second sample as compared to the amount in the first sample is indicative for a candidate compound for the treatment of cancer. Thus, an increased amount indicates that the compound is a potential drug for the treatment of cancer, e.g. for the inhibition of metastasis development after surgical removal of a tumor as described elsewhere herein. Such a compound can be subjected to further tests in order to assess whether it allows for the treatment of cancer.

For example, an increase of the amount of the marker nANGPTL4 (or a variant thereof) in the second sample compared to the amount in the first sample of at least 10%, 30%, 50%, or 100% indicates that the test compound is a candidate compound for the treatment of cancer.

Also preferably, a decreased amount or an unchanged amount of the marker in the second sample as compared to the amount in the first sample indicates that the compound is not a compound for the treatment of cancer.

The present invention also relates to the in vitro use of a detection agent for an N-terminal fragment of an angiopoietin like 4 (ANGPTL4) polypeptide (or a variant thereof) for identifying a candidate compound for the treatment of cancer.

Preferably, the detection agent is used in a first and a second sample of a subject as described herein above in connection with the method for identifying a candidate compound for the treatment of cancer.

Preferably, a “detection agent” in accordance with the present invention relates to an agent which specifically binds the N-terminal fragment of an angiopoietin like 4 (ANGPTL4) polypeptide (or a variant thereof). Examples of “binding agents” or “agents” are antibody, antibody fragment, peptide, peptide nucleic acid (PNA) or chemical compound. A preferred agent is an antibody which specifically binds to the biomarker to be measured. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity (i.e. antigen-binding fragments thereof). Preferably, the antibody is a polyclonal antibody. More preferably, the antibody is a monoclonal antibody (or an antigen binding fragment thereof).

All references cited in this specification are herewith incorporated by reference with respect to their entire disclosure content and the disclosure content specifically mentioned in this specification.

The Figures show:

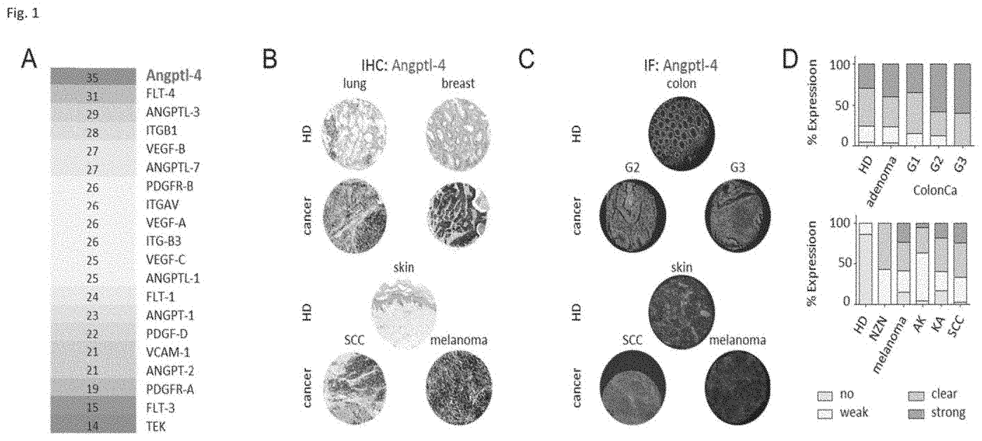

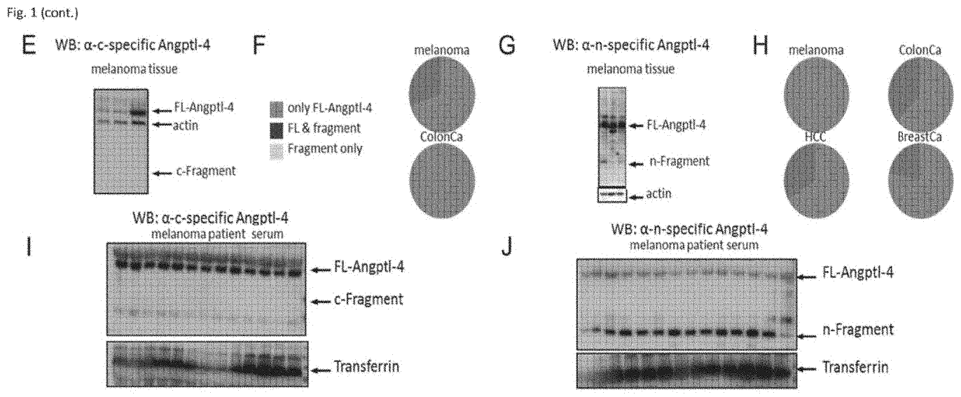

FIG. 1: Identifying ANGPTL-4 and its cleavage parts in human primary tumors and serum. A Analysis of open source Oncomine data, including 49 studies with 13.139 patients with different tumor entities, identified ANGPTL-4 as one of the most upregulated genes. B Immunohistochemistry staining using a C-terminal-specific antibody in TMAs of lung cancer, breast cancer, SCC and melanoma with the corresponding healthy tissue. C Immunofluorescence staining using an N-terminal-specific antibody in TMAs of colon cancer, SCC and melanoma with the corresponding healthy tissue. D Percentage of tumor samples expressing ANGPTL-4 in TMAs (colon cancer n=30 in all entities, healthy skin n=7, compound nevus n=7, actinic keratosis n=120, SCC n=120, melanoma n=150). E Western blot of human melanoma tissue with C-terminal specific antibody. F Analysis of Western blots detecting flANGPTL-4 or cleavage parts in melanoma (n=6) and colon cancer (n=9) G Western blot of human melanoma tissue with N-terminal specific antibody. H Analysis of Western blots detecting flANGPTL-4 or cleavage parts in melanoma (n=6), colon cancer (n=9), breast cancer (n=9) and HCC (n=9). I Western blot of human melanoma patient serum (n=13) using a C-terminal specific antibody. J Western blot of human melanoma patient serum (n=13) using a N-terminal specific antibody.

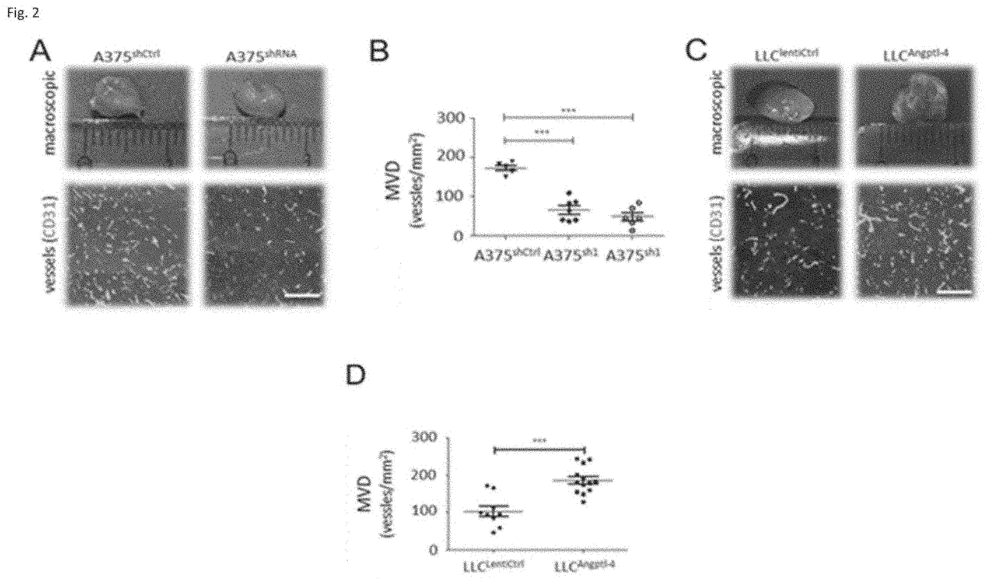

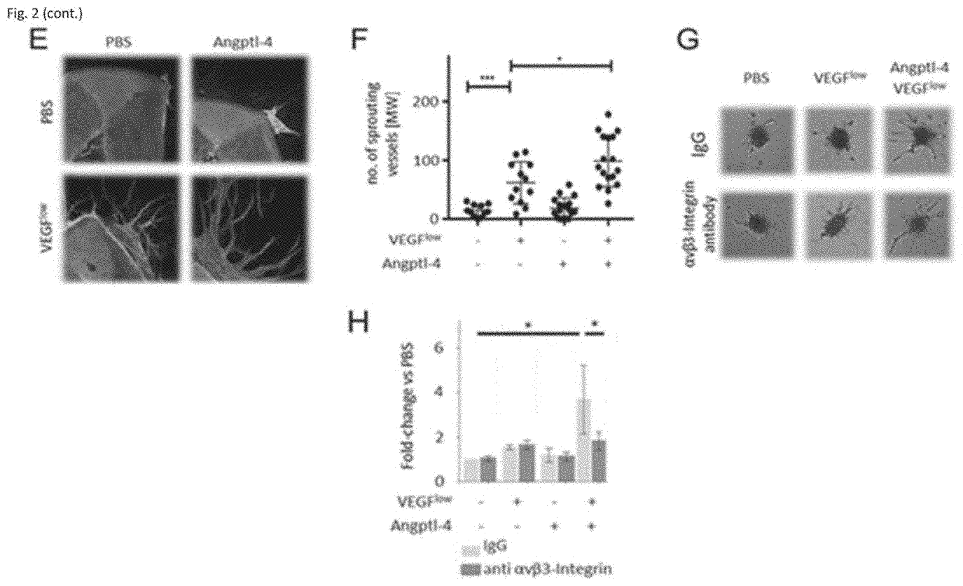

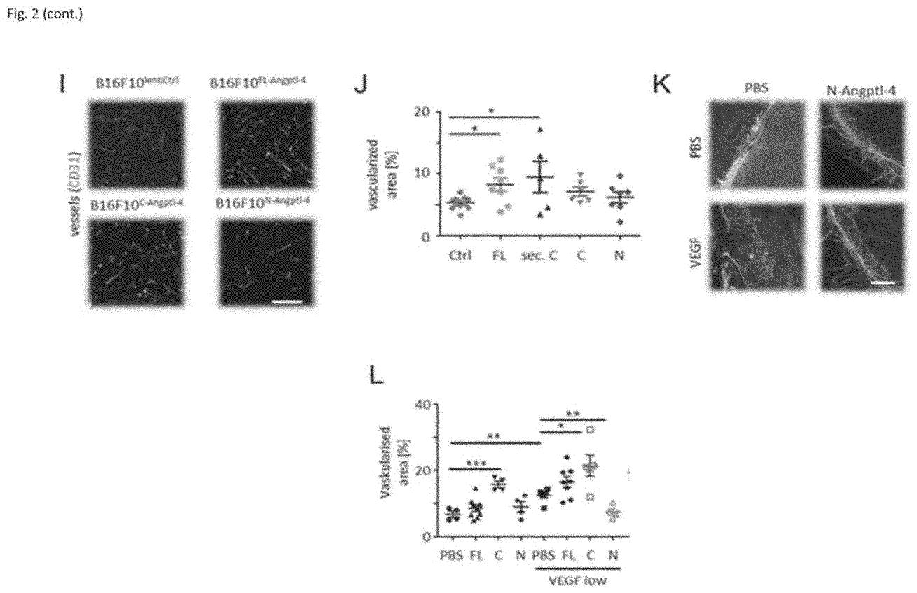

FIG. 2: ANGPTL-4 and its cleavage parts in primary tumor models. A Macroscopic image and IF for CD31 of A375 xenograft primary tumor model with downregulation of ANGPTL-4 (shRNA) compared to control (shcontrol). B Microvessel density of A375 primary tumors downregulating ANGPTL-4 (sh1 and sh2; n=6-7) compared to control (shcontrol; n=5)). C Macroscopic image and IF for CD31 LLC syngenic primary tumor model with upregulation of flANGPTL-4 (LLC Angptl-4) compared to control (LLC Lenticontrol). D Microvessel density of LLC primary tumors upregulating ANGPTL-4 (LLC Angptl-4; n=9) compared to control (LLC Lenticontrol; n=13). E Ex vivo aortic ring assay in which rings were stimulated with either PBS (n=12), VEGF low (n=12), Angptl-4 (n=14) or the combination of VEGF low and Angptl-4 (n=17). F Quantification as number of sprouting vessels per aortic ring. G Representative pictures of sprouting assay; HUVEC were stimulated with either PBS, VEGF low, Angptl-4 or the combination of VEGF low and Angptl-4. Additionally, inhibition with integrin αvβ3-antibody. H Quantification of sprout number per spheroid in all conditions. I B16F10 primary tumor model with tumor cells lentivirally overexpressing flANGPTL-4, nANGPTL-4, cANGPTL-4 in comparison to control tumors. Representative images of CD31 vessel staining in primary tumors. J Quantification of vascularized area in primary tumors. K Cornea pocket assay using flANGPTL-4 (n=9), cANGPTL-4 (n=4), nANGPTL-4 (n=4) in comparison to control (PBS; n=4) and all conditions in combination with VEGF low (n=4; flANGPTL-4 n=8, cANGPTL-4 n=5; nANGPTL-4 n=5), representative images of new vessels infiltrating the cornea upon stimulation. L Quantification of vascularized area in the cornea with or without subcritical dose of VEGF (VEGF low). Data are presented as mean±SD; *p<0.05; **p<0.01, ***p<0,001 by 2-tailed Student's t-Test for in vitro and ex vivo data and as mean±SEM; *p<0.05; **p<0.01, ***p<0,001 by 2-tailed Mann-Whitney U Test for in vivo experiments

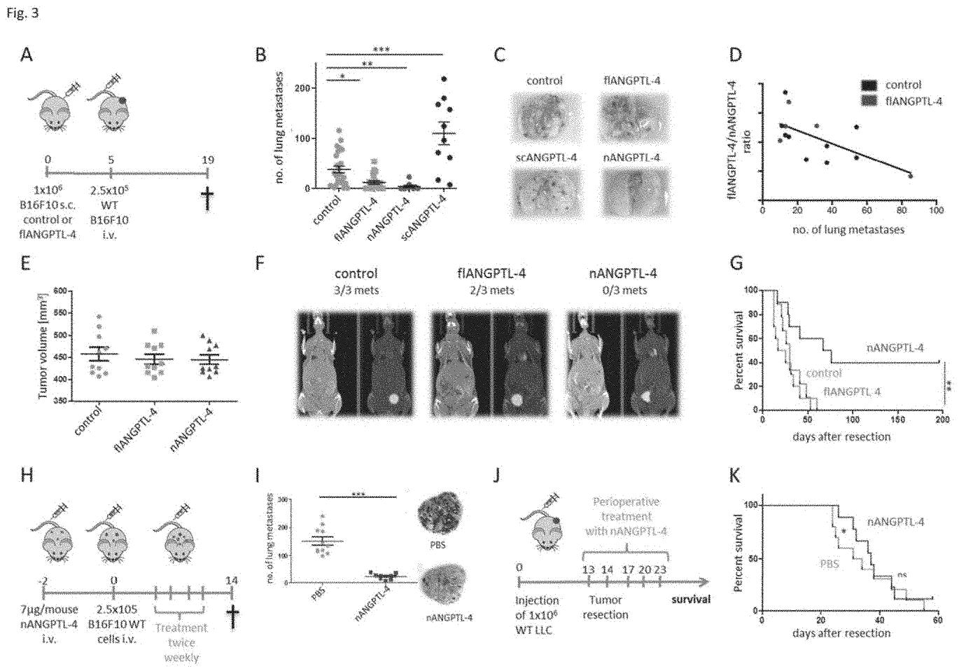

FIG. 3: Analyzing the role of different cleavage fragments of ANGPTL-4 in metastasis models. A Schematic of the modified B16F10 experimental metastasis model. Mice were injected with 1×10⁶ B16 overexpressing or control cells s.c. and five days later with 2.5×10⁵B16F10 WT cells i.v. All mice were sacrificed at day 19. B Quantification of lung metastasis. C Representative images of metastatic lungs. D Mass spectrometric analysis to assess the flANGPTL-4/nANGPTL-4 ratio in control and flANGPTL-4 mice (control group n=9 mice, flANGPTL-4 group n=5 mice). E PET-CT of three representative mice from the LLC spontaneous metastasis model. F Tumor weights at time of resection of the primary tumor (all groups n=10 mice). G Survival curve (based on animal protocol termination criteria) of mice with tumors overexpressing control construct, flANGPTL-4 or nANGPTL-4 (all groups n=10 mice). H Schematic of the B16F10 WT experimental metastasis model and treatment with recombinant mouse nANGPTL-4. I Quantification of lung metastasis and representative images of each group (PBS group n=11 mice, nANGPTL-4 group n=9 mice). J Schematic of the LLC spontaneous metastasis model and perioperative treatment with recombinant mouse nANGPTL-4. K Survival curve of LLC experiment (PBS group n=9 mice, nANGPTL-4 group n=9 mice). Data are presented as mean±SEM; *p<0.05; **p<0.01, ***p<0,001 by 2-tailed Mann-Whitney U Test.

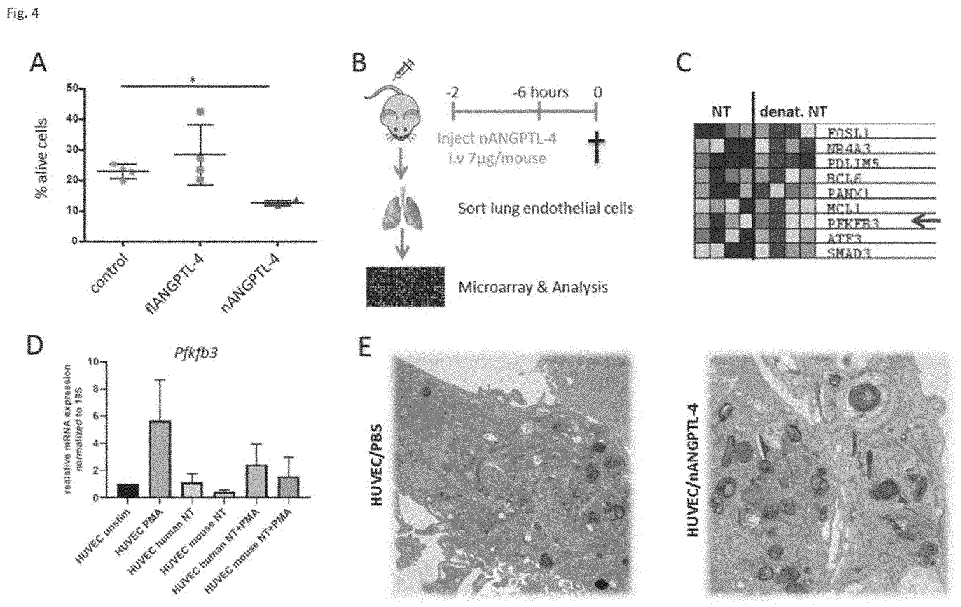

FIG. 4: Effect of nANGPTL-4 on tumor and endothelial cells. A Annexin V staining of overexpressing B16F10 tumor cells and quantitative FACS analysis of live cells (n=4 in all conditions). B Schematic of the treatment of mice used for microarray analysis (n=4 mice per group). C Geneset enrichment analysis of treated mouse lung endothelial cells [denatured nANGPTL-4 compared to functional nANGPTL-4]. D Confirmation of nANGPTL-4 effect on human endothelial cells (HUVEC) stimulated with PMA, mouse nANGPTL-4 or human nANGPTL-4 or both (n=2) E Electron microscopic pictures of HUVEC stimulated with PBS or human nANGPTL-4. Data are presented as mean±SD; *p<0.05; **p<0.01, ***p<0,001 by 2-tailed Student's t-Test

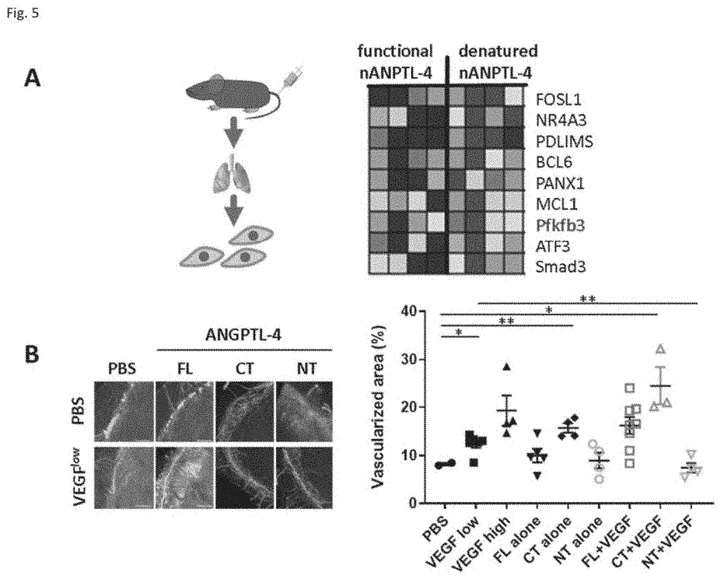

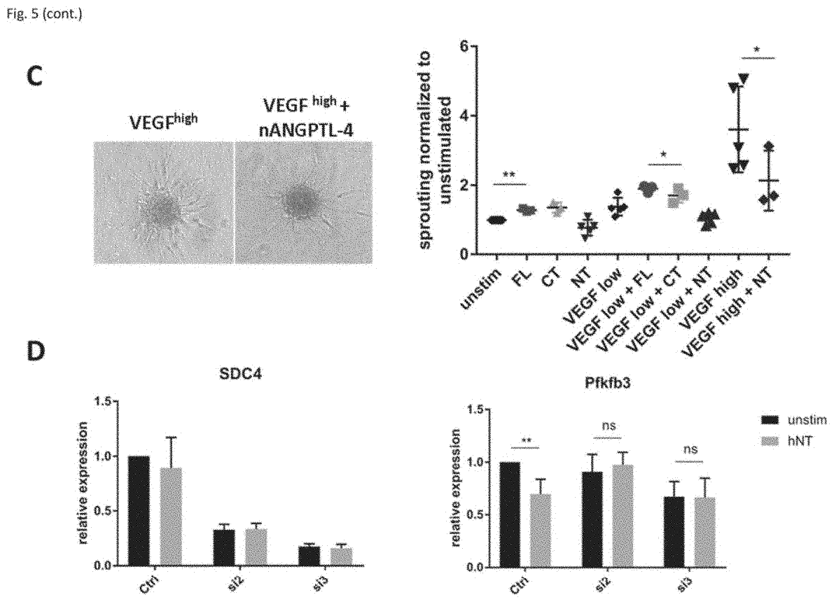

FIG. 5: nANGPTL-4 inhibits metastasis via SDC4-dependent sprouting angiogenesis. A Microarray of sorted mouse lung endothelial cells stimulted with either denatured nANGPTL-4 or functional nANGPTL-4. B Cornea pocket assay using control, flANGPTL-4 and its cleavage products with or w/o VEGFlow. C Sprouting Data of HUVEC stimulated with human recombinant protein (n=3-5). D HUVEC stimulated with nANGPTL-4 and expression levels of Pfkfb3 and SDC4 were assessed via RT-qPCR.

### EXAMPLES

The invention will be merely illustrated by the following Examples. The said Examples shall, whatsoever, not be construed in a manner limiting the scope of the invention.

### Example 1: Identifying ANGPTL-4 and its Cleavage Parts in Human Primary Tumors and Serum

High ANGPTL-4 protein expression revealed in a meta-analyses of 49 different studies with 15 different tumor entities with 13.139 patient samples with a dichotomous read-out for twenty angiogenic factors indicated in most studies (35/49) a poor outcome for the patients (FIG. 1A). Investigating the expression patterns of ANGPTL-4 in different human tumors, we screened its expression in Tissue Microarrays (TMA) of melanoma, cutaneous squamous cell carcinoma (cSCC), colon cancer, breast cancer and lung cancer and compared it with healthy control tissue. Using immunofluorescence (IF) and immunohistochemistry (IHC) with different antibodies, we observed widespread, elevated levels of ANGPTL-4 expression in the majority of the tumor samples compared to the corresponding healthy tissues (FIG. 1A-C). Comparative clinical studies of the IF studies in melanoma, cSCC and colon cancer samples showed an increase in ANGPTL-4 expression levels with tumor progression (FIG. 1D). Extending experiments were aimed to assess which form of ANGPTL-4 (flANGPTL-4, cANGPTL-4 or nANGPTL4) is mainly found in the tumor tissues. To address this question, we performed Western blot analysis using human anti-c- and anti-n-specific ANGPTL-4 antibodies on melanoma, colon cancer, hepatocellular carcinoma (HCC), as well as breast cancer tissues (exemplary of melanoma FIGS. 1E & 1G). flANGPTL-4 was detectable in all samples with the c-specific antibody (FIG. 1F) as well as with the n-specific antibody (FIG. 11I). In no samples was only cleaved ANGPTL-4 form detectable. FlANGPTL-4 and the cleavage products (cANGPLT-4 or nANGPTL-4) were expressed in a minority of samples. We further analyzed the levels of n-, c- and flANGPTL-4 in serum samples of melanoma patients (FIGS. 1 I & 1J, longer exposure time vs. 1I). Mainly nANGPTL-4 was detectable in the serum of the patients. flANGPTL-4 was significantly less compared to nANGPTL-4 (FIG. 1I) and cANGPLT-4 was less than flANGPTL-4 (FIG. 1J).

### Example 2: ANGPTL-4 and its Cleavage Parts in Primary Tumor Models

To study the contribution of ANGPTL-4 in tumor progression, we modified the expression of ANGPTL-4 expression in different established human and mouse cell line models. In a xenograft melanoma mouse model with A375 tumors with intrinsically high levels of ANGPTL-4, loss of ANGPTL-4 expression with a lentiviral approach resulted in reduced vascularity of the tumors (FIG. 2A) with reduced CD31-expressing vessels (FIG. 2B). In a syngeneic lung carcinoma model with LLC tumor cells, increased expression of ANGPLT-4 led to macroscopically better vascularized tumors (FIG. 2C) with increased CD31-expressing vessels in the tumors (FIG. 2D). In order to investigate, how flANGPTL-4 enhances angiogenesis, the aortic ring assay as well the spheroid sprouting assays were performed. FlANGPTL-4 mono-stimulation did not alter angiogenesis in the ex vivo aortic ring assay. Yet, flANGPTL-4 enhanced low dose VEGF-induced angiogenesis (FIGS. 2E and F). Similarly, mono-stimulation of the EC with flANGPTL-4 did not alter sprouting angiogenesis in the in vitro sprouting assay. FlANGPTL-4 enhanced VEGF low dose sprouting angiogenesis in the spheroid sprouting assay, too. Pretreatment of the EC with an integrin αvß3 blocking antibody diminished this effect (FIGS. 2G and H), suggesting that the pro-angiogenic effect of flANGPTL-4 requires VEGF and is integrin-dependent. Next, we investigated the role of the different ANGPTL-4 forms (nANGPTL-4, cANGPTL-4 and flANGPTL4) in another syngeneic mouse melanoma tumor model. Growth of the different primary tumors was observed and plotted as growth curve (data not shown). Here, we observed a strong growth advantage of B16F10 cells overexpressing cANGPTL-4 in early stages of tumor induction. MVD analyses, which were assessed in tumors of the same size, showed a significant increase in cells overexpressing flANGPTL-4 and cANGPTL-4 (FIGS. 2I and J). To investigate the relevance of the cleavage parts (cANGPTL-4 and nANGPTL-4) in another angiogenesis model, cornea pocket assays were performed. The cornea is an avascular tissue in the eye which is nourished by the tear fluid. Only the limbus is vascularized, from which in pathological conditions vessels can grow out. Inserting a small pellet containing the pro- or antiangiogenic agent into a pre-made pocket in the cornea shows outgrowing microvessels within one week of exposure. All vessels growing inside the cornea are newly formed vessels from the pre-existing limbus vessels. Corneas were harvested and stained for CD31 in order to analyze the microvessel density (FIGS. 2L and K). We observed a strong increase in MVD in this experiment when stimulated with fl- or cANGPTL-4. nANGPTL-4 alone did not show any angiogenic effect compared to control, but reduced VEGF-induced sprouting in this assay.

### Example 3: Analyzing the Role of Different Cleavage Fragments of ANGPTL-4 in Metastasis Models

So far, most mouse metastasis models do not allow to decipher the effect of primary tumor cell secreted factors on metastasis as metastasis and primary tumors are not present in the mouse at the same timepoints. Therefore, we developed a new melanoma mouse model which allowed to study primary tumor and metastasis formation at the same time. First, we injected 1×10⁶ B16F10 tumor cells subcutaneously into the flank of C57Bl/6 mice. Some of these tumor cells were lentivirally modified to overexpressed flANGPTL-4, cANGPTL4 or nANGPTL-4. Five days later, when initial macroscopic tumor formation was visible, intravasation of tumor cells was mimicked via intravenous injection of 2.5×10⁵ wildtype B16F10 tumor cells. Wildtype cells were used for tail vein injection to exclude effects of ANGPTL-4 secreted by the circulating tumor cells. Mice were sacrificed 14 days after tail vein injection (total day 19) and the number of lung metastases was analyzed (FIG. 3A). When analyzing the lungs, we observed a significant reduction of lung metastasis when the primary tumors overexpressed flANGPTL-4 or nANGPTL-4. In contrast, overexpression of cANGPTL-4 by the primary tumor cells increased the number of lung metastasis compared to control (FIGS. 3B and C). When using this model, a substantial deviation between the mice occurred, e.g. in control group ranging from 1 to over 100 lung metastases. Considering the substantial deviation, we established a mass spectrometry analysis for murine ANGPTL-4 from mouse serum to check the levels of ANGPTL-4 in those mice. The ratio between the full-length protein and the N-Terminal part was determined in this analysis. Here we discovered that the nANGPTL-4/fl-ANGPTL-4 ratio inversely correlated with the number of metastasis (R=0.3972, P=0.0157). Mice from the control group (black symbols) and mice from the flANGPTL-4 group (red symbols) were included. The effect was detectable in both groups (FIG. 3D). Next, a lung carcinoma metastasis model was used in which lung metastasis form after primary tumor resection. To have comparable groups, we injected in all mice 1×10⁶ LLC tumor cells, either with a control construct or fl- or nANGPTL-4 overexpression and resected the tumors at the same size (FIG. 3E). Following resection, the mice underwent CT or PET-CT scans weekly for three consecutive weeks. Representative PET-CT scans are shown in FIG. 3F, indicating the location of the metastasis (red circles). All mice in the control group developed lung and/or liver metastasis. In flANGPTL-4, 2 out of 3 mice had metastasis and in nANGPTL-4 mice no metastases were detectable three weeks after resection. CT/PET-CT scans were done for three mice per group. All the mice were killed when predefined termination criteria were fulfilled (FIG. 3G). This curve shows a significant survival benefit for the mice overexpressing nANGPTL-4 in their tumors. To evaluate therapeutic potential of nANGPTL-4, we produced the murine N-Terminal fragment as recombinant protein. Mice were treated two days before intravenous tumor cell injection with 7 μg/mouse of protein. Thereafter, 2.5×10⁵ B16F10 tumor cells were injected into the tail vein. Following tumor cell injection, the mice were treated twice weekly with the above-mentioned amount of protein per injection (FIG. 3H). The mice were sacrificed two weeks after tail vein injection and lung metastases were evaluated. Here we saw a significant reduction of lung metastases upon treatment with recombinant murine nANGPTL-4 (FIG. 3I). Additionally, we tested the effect of nANGPTL4 on spontaneous metastasis using the LLC model. After having injected the tumor cells, we treated the mice with either PBS or recombinant mouse nANGPTL-4. The first injection was administered one day before resection of the primary tumor and three doses were given in an adjuvant manner after resection (FIG. 3J). Again, the readout of this experiment was the ‘survival’ of the mice when they had to be sacrificed due to predefined termination criteria. Treated mice had a median survival of 32 days in the PBS group and 37.5 days in the group treated with nANGPTL-4. Nonetheless, 38 days after tumor resection the curves of the two groups merged and were not significantly different anymore (FIG. 3K). Taken together, these data suggest that infusion of nANGPTL-4 may be a viable therapeutic option against aggressive metastatic cancers.

### Example 4: Effect of nANGPTL-4 on Tumor and Endothelial Cells

Next, we set out experiments to address the question, how nANGPTL-4 mechanistically regulates metastasis formation. ANGPTL-4 is a key regulator of anoikis. We therefore checked if alteration of ANGPTL-4 expression (flANGPTL-4 or its fragments) affected anoikis in vitro. To do so, we plated B16F10 overexpressing cells under anoikis conditions for 48 h, stained the cells with Annexin V and implemented a quantitative FACS analysis to test the number of living cells. We observed in this experiment a significant reduction of living cells (Annexin V-FxCycle) in B16F10 overexpressing nANGPTL-4, (FIG. 4A), indicating that these cells have a lesser chance to survive in the circulation compared to control or flANGPTL-4 overexpressing cells. Extending experiments were performed to unravel if flANGPTL-4, nANGPTL-4 or c-ANGPTL-4 alter angiogenesis as an important step for macroscopic metastatic colony formation. A significant decrease of MVD was observed in a model with LLC tumors and two consecutive treatments with recombinant murine nANGPTL-4. Aiming to decipher the molecular mechanism for the observed antiangiogenic effect, microarrays of FACS-isolated EC, which had been treated with murine nANGPTL-4 or heat-inactivated murine n-ANGPTL-4 were performed (FIG. 4B). Analysis of the array revealed a strong downregulation of Pfkfb3, a potent regulator of glycolysis in endothelial cells (FIG. 4C). Endothelial cells rely on glycolysis rather than on oxidative phosphorylation for ATP production. The loss of the glycolytic activator PFKFB3 in endothelial cells impaired vessel formation and sprouting angiogenesis (Mandard et al., Cantelmo et al.). Next, we aimed to confirm the downregulation of Pfkfb3 in vitro upon stimulation with nANGPTL-4 in HUVEC cells in vitro. To further expose the cells to stress conditions as it may occur physiologically under metastatic conditions, the cells were stimulated after nANGPTL-4 treatment with Phorbol 12-myristate 13-acetate (PMA) for 1 h. Upon stimulation with PMA, we observed a substantial upregulation of Pfkfb3. When stimulating the cells with either recombinant human or mouse nANGPTL-4, we identified a reduction of Pfkfb3 expression (FIG. 4D). In order to investigate the effect of nANGPTL4 on the microanatomy of HUVEC, we took electron microscopic images of stimulated cells. Here we observed an increased number of microvesicular bodies, suggesting that the cells underwent a potent cell stress when stimulated with nANGPTL-4 (FIG. 4E).

### Example 5: nANGPTL-4 Inhibits Metastasis Via SDC4-Dependent Sprouting Angiogenesis

Aiming to identify the molecular signaling targets of nANGPTL-4, mice were treated with nANGPTL-4 and compared with mice treated with heat-inactivated nANGPTL-4 (FIG. 5A). EC of both groups were isolated out of the lungs and compared with microarray technique (FIG. 5A). Among the top regulated genes, downregulation of PFKFB3 was observed. PFKFB3 is a key molecule for sprouting angiogenesis of EC (de Bock et al., Cell 2013). Therefore, we studied the effect of nANGPTL-4 and other variants of ANGPTL-4 in the cornea pocket assay in vivo (FIG. 5B). Here, treatment with nANGPTL-4 only, did not affect sprouting angiogenesis (compare PBS vs. NT alone). In contrast, the C-fragment of ANGPTL-4 enhanced sprouting angiogenesis significantly (compare PBS vs. CT alone). Strikingly, nANGPTL-4 inhibited VEGF-induced angiogenesis (compare VEGF low vs NT+VEGF). Similar results were obtained in the spheroid sprouting assay (FIG. 5C). While mono-stimulation with nANGPTL-4 had no effect on angiogenesis (compare unstim. vs. NT), nANGPTL-4 inhibited VEGF-induced sprouting angiogenesis (compare VEGF high vs VEGF high+NT). C- as well as FL-ANGTL-4 enhanced angiogenesis (FIG. 5C and data not included). This experiments show for the first time, that ANGPTL4 can have two opposing functions. It can be pro- or antiangiogenic. Interestingly, Cazes et al. observed an anti-angiogenic effect in their spheroid sprouting experiments (Cazes et al., 2006). Apparently, the full-length protein was tested. In contrast, this effect was not observed for the full-length protein in the studies underlying the present invention.

Recently, syndecans 2 and 4 (SDC2 and SDC4) were identified as the receptors of nANGPTL-4-induced signaling in a systematic screen using a pull-down and mass spectrometry approach (Kirsch et al., Dev. Cell 2017). We isolated endothelial cells (EC) of metastatic lung and compared it with non-metastatic lung. Here we observed that expression of SDC4 but not SDC2 was significantly upregulated in the EC of metastatic lungs (data not shown). Extending experiments with siRNA, proove that treatment of EC with nANGPTL-4 reduces expression PFKFB3, a master regulator of sprouting angiogenesis (de Bock et al., Cell 2013). This inhibitory effect is lost, when expression of SDC4 is downregulated with siRNA (FIG. 5D).

### CONCLUSIONS

Angiopoietin-like 4 (ANGPTL-4) is a secreted glycoprotein for which conflicting pro-tumorigenic and antitumorigenic functions have been reported. We observed that ANGPTL-4 is proteolytically cleaved upon secretion into two fragments, a C-terminal and an N-terminal fragment. The uncleaved variant of ANGPTL-4 was detectable in every single tumor sample of various cancer entities (melanoma, breast cancer, colon cancer and hepatocellular carcinomas). In these tissues the fragments of ANGPTL-4 were only detectable in a minority of the samples. Using a broad array of tumor models, we discovered that nANGPTL-4 did not affect primary tumor growth, but profoundly inhibited distant site metastasis formation and enhanced overall survival in mice. Therefore, a therapeutic concept of surgical resection of the primary tumor combined with nANGPTL-4 treatment thereafter is promising. These findings are of major importance as i.) it has a direct therapeutic value using nANGPTL-4 as new anti-metastatic drug, ii.) it revitalize old concepts of primary tumors suppressing metastases through the systemic release of metastasis-inhibiting cytokines, and iii.) it is the first protein with a pro- and anti-(angiogenic) effect and therefore has more than the general on-/off-phenomena which can be found in different proteins.

In summary, the results show that nANGPTL-4 has a strong therapeutic potential to inhibit metastasis. By specifically suppressing metastatic outgrowth, the systemic disease cancer can be limited to a local tumor, for which various treatment options are already established.

### REFERENCES

Tan et al.: Emerging roles of angiopoietin-like 4 in human cancer. Mol Cancer Res. 2012; 10:677-688
Paglia et al.: Potential Role of ANGPTL4 in the Cross Talk between Metabolism and Cancer through PPAR Signaling Pathway. PPAR Res. 2017; 2017:8187235
Zhang et al.: Angiopoietin-like 4 promotes osteosarcoma cell proliferation and migration and stimulates osteoclastogenesis. BMC Cancer (2018) 18:536
Chiang A C, Massagué J.: Molecular basis of metastasis. N Engl J Med, 359: 2814-23, 2008.
Augustin H G, Koh G Y:. Organotypic vasculature: From descriptive heterogeneity to functional pathophysiology. Science, 357: eaa12379, 2017.
Folkman J: Tumor angiogenesis: Therapeutic implications. N Engl J Med, 285: 1182-6. 1971.
Vasudev et al.: Anti-angiogenic therapy for cancer: current progress, unresolved questions and future directions. Angiogenesis, 17: 471-94, 2014.
Augustin H G, et al.: Control of vascular morphogenesis and homeostasis through the angiopoietin-Tie system. Nat Rev Mol Cell Biol, 10: 165-77, 2009.
Kirsch N, et al.: Angiopoietin-like 4 is a Wnt signaling antagonist that promotes LRP6 turnover. Dev Cell, 43: 71-82, 2017.
Zhu P, et al.: Angiopoietin-like 4: A decade of research. Biosci Rep, 2: 211-9, 2012.
Padua, D, et al.: TGFβ primes breast tumors for lung metastasis seeding through Angiopoietin-like 4. Cell, 133: 66-77, 2008.
Tian L, et al.: Activating peroxisome proliferator-activated receptor gamma mutant promotes tumor growth in vivo by enhancing angiogenesis. Cancer Res.: 69: 9236-44, 2009.
Zhu, P et al. Angiopoietin-like 4 protein elevates the prosurvival intracellular O₂—:H₂O₂ ratio and confers anoikis resistance to tumors. Cancer Cell, 19: 401-15, 2011.
Huang, R L et al.: ANGPTL4 modulates vascular junction integrity by integrin signaling and disruption of intercellular VE-cadherin and claudin-5 clusters. Blood, 118: 3990-4002, 2011.
Hu K, et al.: Hypoxia-inducible factor 1 upregulation of both VEGF and ANGPTL4 is required to promote the angiogenic phenotype in uveal melanoma. Oncotarget, 7: 7816-28, 2016.
Liao Y H, et al.: Epidermal growth factor-induced ANGPTL4 enhances anoikis resistance and tumour metastasis in head and neck squamous cell carcinoma. Oncogene, 36: 2228-42, 2017.
Baba K, et al.: Hypoxia-induced ANGPTL4 sustains tumour growth and anoikis resistance through different mechanisms in scirrhous gastric cancer cell lines. Sci Rep, 7: 11127, 2017.
Zhang T, et al.: Angiopoietin-like 4 promotes osteosarcoma cell proliferation and migration and stimulates osteoclastogenesis. BMC Cancer, 18: 536, 2018.
Chen J W, et al.: Knockdown of angiopoietin-like 4 inhibits the development of human gastric cancer. Oncol Rep, 39: 1739-46, 2018.
Galaup A, et al.: Angiopoietin-like 4 prevents metastasis through inhibition of vascular permeability and tumor cell motility and invasiveness. Proc Natl Acad Sci USA, 103: 18721-6, 2006.
Cazes A, et al.: Extracellular matrix-bound angiopoietin-like 4 inhibits endothelial cell adhesion, migration, and sprouting and alters actin cytoskeleton. Circ Res, 99: 1207-15, 2006.
Ito Y, et al.: Inhibition of angiogenesis and vascular leakiness by angiopoietin-related protein 4. Cancer Res, 63: 6651-7, 2003.
Gomez Perdiguero E, et al.: ANGPTL4-αvβ3 interaction counteracts hypoxia-induced vascular permeability by modulating Src signaling downstream of vascular endothelial growth factor receptor 2. J Pathol, 240: 461-71, 2016
Okochi-Takada E, et al.: ANGPTL4 is a secreted tumor suppressor that inhibits angiogenesis. Oncogene, 33: 2273-8, 2014.
Ng K T, et al.: Clinical relevance and therapeutic potential of angiopoietin-like protein 4 in hepatocellular carcinoma. Mol Cancer, 13:196, 2014
Teo, Z et al.: Elevation of adenylate energy charge by angiopoietin-like 4 enhances epithelial-mesenchymal transition by inducing 14-3-3γ expression. Oncogene 36: 6408-19, 2017.
Li L, et al.: Angiopoietin-like 4 increases pulmonary tissue leakiness and damage during influenza pneumonia. Cell Rep, 10: 654-663 (2015).
Mandard S, et al.: The direct peroxisome proliferator-activated receptor target fasting-induced adipose factor (FIAF/PGAR/ANGPTL4) is present in blood plasma as a truncated protein that is increased by fenofibrate treatment. J Biol Chem, 279: 34411-20, 2004.
Cantelmo A R, et al.: Inhibition of the glycolytic activator PFKFB3 in endothelium induces tumor vessel mormalization, impairs metastasis, and improves chemotherapy. Cancer Cell, 30: 968-85, 2016.
De Bock K, et al.: Role of PFKFB3-driven glycolysis in vessel sprouting. Cell., 154: 651-63, 2013

1. A method for the treatment of cancer in a subject in need thereof, the method comprising administering to the subject a therapeutically effective amount of an N-terminal fragment of an angiopoietin like 4 (ANGPTL4) polypeptide, or a therapeutically active variant thereof.

2. The method of claim 1, wherein the treatment of cancer is the inhibition of metastasis development.

3. The method of claim 2, wherein the inhibition of metastasis development is the inhibition of metastasis development after surgical removal of a tumor.

4. The method of claim 3, wherein the N-terminal fragment, or therapeutically active variant thereof, is administered perioperatively.

5. The method of claim 3, wherein the N-terminal fragment, or therapeutically active variant thereof, is administered before and/or after surgical removal of the tumor.

6. The method of claim 3, wherein the tumor is a primary tumor and wherein the subject has not developed metastases at the time of the surgical removal of the tumor.

7. The method of claim 1, wherein the N-terminal fragment, or therapeutically active variant thereof, is administered intravenously or intraperitoneally.

8. The method of claim 1, wherein the subject is a human subject and wherein the angiopoietin like 4 (ANGPTL4) polypeptide is human ANGPTL4.

9. The method of claim 1, wherein the N-terminal fragment, or therapeutically active variant thereof, is selected from
a) a polypeptide comprising a sequence as shown in SEQ ID NO: 1, SEQ ID NO 2, SEQ ID NO 7, or SEQ ID NO 8,
b) a subfragment of the polypeptide of a), wherein the subfragment has a length of at least 50 amino acids, and
c) a polypeptide having at least 60%, at least 70%, at least 80%, at least 85%, at least 90%, at least 95%, at least 97%, at least 98% or at least 99% sequence identity to the polypeptide of a) or the subfragment of b).

10. The method of claim 1, wherein the N-terminal fragment, or therapeutically active variant thereof, is capable of
i. decreasing the activity of lipoprotein lipase,
ii. binding to Syndecan-4, and/or
iii. forming oligomers.

11. The method of claim 1, wherein the cancer is selected from melanoma, breast cancer, colorectal cancer, ovarian cancer, renal cancer, gastrointestinal cancer, lung cancer, primary cutaneous lymphomas and hepatocellular carcinoma.

12. A method for treating cancer in a subject in need thereof, the method comprising administering to the subject a therapeutically effective amount of an agent which increases the amount of an N-terminal fragment of an angiopoietin like 4 (ANGPTL4) polypeptide.

13. A method for identifying a candidate compound for the treatment of cancer, comprising
a) determining the amount of an N-terminal fragment of an angiopoietin like 4 (ANGPTL4) polypeptide in a first sample from a subject, wherein said first sample has been obtained from the subject prior contacting the subject with the candidate compound,
b) determining the amount of the N-terminal fragment of an angiopoietin like 4 (ANGPTL4) polypeptide in a first sample from a subject, wherein said second sample has been obtained after contacting the subject with the candidate compound, and
c) comparing the amount of the N-terminal fragment of an angiopoietin like 4 (ANGPTL4) polypeptide in the second sample to the amount in the first sample, wherein an increased amount in the second sample as compared to the amount in the first sample is indicative for a candidate compound for the treatment of cancer.

14. The method of claim 13, wherein the sample is blood, serum or plasma sample.

15. The method of claim 13, wherein the method is an in vitro method.
